(19) United States
(12) Patent Application Publication
(10) Pub. No.: US 2020/0150117 A1
(43) Pub. Date: May 14, 2020

(54) IMMUNO-ONCOLOGY FOR THE TREATMENT OF CANCER

(71) Applicant: Oncimmune Germany GmbH, Dortmund (DE)

(72) Inventors: Hans-Dieter ZUCHT, Hannover (DE); Petra BUDDE, Dortmund (DE); Peter SCHULZ-KNAPPE, Hemmingen (DE)

(21) Appl. No.: 16/625,898

(22) PCT Filed: Jun. 23, 2018
(86) PCT No.: PCT/EP2018/066840

§ 371 Date: Dec. 23, 2019

Related U.S. Application Data
(60) Provisional application No. 62/524,220, filed on Jun. 23, 2017.

(51) Int. Cl. G01N 33/564; G01N 33/574

(52) U.S. Cl. CPC G01N 33/564; G01N 2800/52; G01N 33/57434

(57) ABSTRACT

The invention relates to a method of identifying a tumor-associated antigen (TAA) for prostate cancer. Moreover, the invention provides a method of identifying a TAA as a marker for prostate cancer vaccination response. Particularly, a method of identifying and treating a prostate cancer patient with PROSTVAC therapy or for vaccination with a prostate antigen.

### BACKGROUND OF THE INVENTION

According to the World Health Organization (WHO) cancer is one of the leading causes of morbidity and mortality worldwide, with approximately 14 million new cases and 8.2 million cancer deaths in 2012 worldwide (Ferlay et al., 2015). An estimated 21.6 million new cancer cases are predicted for 2030 (an increase of 53 percent from 2012.

The economic impact of cancer is significant is increasing. In the US the total annual economic cost of cancer in 2010 were estimated at approximately US$ 1.16 trillion.

According to GLOBOCON the four most commonly new diagnosed cancer types in 2012 were lung (1.82 million), breast (1.67 million), colorectal (1.36 million), and prostate cancer (1.1 million) (Ferlay et al., 2015).

There are many types of cancer treatment, which depend on the cancer type. These include classical treatments such as surgery with chemotherapy and/or radiation therapy or hormone therapy. New therapies aim to directly target the tumor or to inhibit the growth of the tumor with tyrosine kinase inhibitors, monoclonal antibodies, and proteasome inhibitors.

Despite improvements in current therapies, the low survival rates of cancer are due to inadequate early diagnosis, resistance to current therapies, and ineffective treatment. Thus, alternative treatment approaches are desperately needed for cancer.

In contrast to targeting cancer-specific oncogenes, which promote survival and metastasis of cancer, the primary goal of cancer immunotherapy is to stimulate the human immune system to identify and destroy developing tumors.

The concept of cancer immunotherapy is based on the finding that many tumor cells express abnormal proteins and molecules, which in theory should be recognized by the immune system. Proteins, which are present in the tumor and elicit an immune response, are called tumor-associated antigens (TAA). The group of TAA comprises mutated proteins, overexpressed or aberrantly expressed proteins, proteins produced by oncogenic viruses, germline-expressed proteins, glycoproteins or proteins, which are produced in small quantities or are not exposed to the immune system. The immune response to TAA includes cellular processes as well as the production of antibodies against TAA.

However, forcing immune cells to recognize the tumor as foreign is proving to be much more difficult than anticipated. This is because the tumor effectively suppresses immune responses by activating negative regulatory pathways. These negative regulatory pathways are called immune-checkpoints, which under normal physiologic conditions; maintain a careful balance between activating and inhibitory signals thereby protecting the normal tissue from damage.

Collectively, these findings have led to different immunotherapeutic approaches including active, passive and immunomodulatory approaches.

Active immunotherapies directly stimulate the immune system to target tumors using inflammatory factors such as cytokines or therapeutic cancer vaccines.

For example, PROSTVAC cancer vaccination is intended to trigger a specific and targeted immune response against prostate cancer. PROSTVAC is a virus-based vaccine that carries the tumor-associated antigen PSA/KLK3 (prostate-specific antigen) along with three natural human immune-enhancing costimulatory molecules collectively designated as TRICOM (LFA3, ICAM1, and B7.1/CD80). The PSA-TRICOM vaccines infects antigen-presenting cells (APCs) and generate proteins that are expressed on the surface of the APCs by major histocompatibility complex (MHC) proteins. This leads to T-cell activation.

PROSTVAC is currently tested in phase 3 clinical trials for treating minimally symptomatic metastatic prostate cancer (mCRPC). Prior phase 2 clinical studies showed that patients who received PROSTVAC had a median overall survival that was 8.5 months longer than the control group (25.1 versus 16.6 months) and a 44% reduction in the risk of death (stratified log-rank P=0.0061). PROSTVAC was generally well tolerated, with the most common side effects including injection site reactions, fever, fatigue, and nausea (Kantoff et al., 2017).

Passive immunotherapies usually utilize monoclonal antibodies targeting immune checkpoint molecules. The cytotoxic T-lymphocyte-associated antigen 4 (CTLA-4) and programmed death 1 (PD-1) immune checkpoints are negative regulators of T-cell immune function, when bound to their respective ligands CD80/86 and programmed cell-death ligand 1 and 2 (PDL1/PDL2).

In addition to anti-CTLA4 and anti-PD1/PDL1 antibodies, drugs targeting other checkpoints such as lymphocyte activation gene 3 protein (LAG3), T cell immunoglobulin mucin 3 (TIM-3), and IDO (Indoleamine 2,3-dioxygenase) are in development.

Inhibition of checkpoint inhibitors, resulting in increased activation of the immune system, has led to new immunotherapies for melanoma, non-small cell lung cancer, and other cancers (Buchbinder and Desai, 2016).

Ipilimumab, an inhibitor of CTLA-4, is approved for the treatment of advanced or unresectable melanoma.

Nivolumab and pembrolizumab, both PD-1 inhibitors, are approved to treat patients with advanced or metastatic melanoma and patients with metastatic, refractory non-small cell lung cancer.

Anti-PDL1 inhibitor avelumab has received orphan drug designation by the European Medicines Agency for the treatment of gastric cancer in January 2017. The US Food and Drug Administration (FDA) approved it in March 2017 for Merkel-cell carcinoma, an aggressive type of skin cancer.

Despite the fact that checkpoint inhibitors, demonstrated clinical efficacy across multiple cancer types, checkpoint inhibitor drugs are not effective against all cancer types, nor in every patient within a cancer type (Brahmer et al., 2012).

In addition, compared to cancer vaccination strategies, checkpoint inhibitors can induce severe immune-related adverse events (irAE). The main side effects include diarrhea, colitis, hepatitis, skin toxicities, arthritis, diabetes, endocrinopathies such as hypophysitis and thyroid dysfunction (Spain et al., 2016).

Therefore, biomarkers are needed to predict both clinical efficacy and toxicity. Such biomarkers may guide patient selection for both monotherapy and combination therapy (Topalian et al., 2016).

There are apparent differences between the CTLA4 and PD1 pathways of the immune response. CTLA4 acts more globally on the immune response by stopping potentially autoreactive T cells at the initial stage of naive T-cell activation, typically in lymph nodes. The PD-1 pathway regulates previously activated T cells at the later stages of an immune response, primarily in peripheral tissues (Buchbinder and Desai, 2016).

Substantial efforts have been undertaking to identify biomarkers for predicting which patient will respond best to immune checkpoint inhibition.

Given the mechanism of action of inhibiting the PD1 pathway, several studies have evaluated the expression of the PDL1 ligand in the tumor as a biomarker of clinical response. However, differences regarding the predictive value of PDL1 expression have been found. This limits the current use of PDL1 as a biomarker for predicting clinical response. The differences in the utility of PDL1 as biomarker may be caused by differences in the assay type used in different studies and by variable expression of PDL1 during therapy (Manson et al., 2016).

Since checkpoint inhibition is typically viewed as enhancing the activity of effector T cells in the tumor and tumor environment, other biomarker approaches have focused on identifying TAA recognized by T cells. However, this approach is limited to exploratory analyses and is not practical in a routine laboratory setting because it requires patient-specific MHC reagents (Gulley et al., 2014).

A largely overlooked immune cell type in the context of immunotherapies are B cells, which can exert both anti-tumor and tumor-promoting effects by providing co-stimulatory signals and inhibitory signals for T cell activation, cytokines, and antibodies (Chiaruttini et al., 2017). B-cells produce anti-tumor antibodies, which can potentially mediate antibody-dependent cellular cytotoxicity (ADCC) of tumor cells. It is well established that many cancer types induce an antibody response, which can be used for diagnostic purposes. Although some cancer patients show an antibody response to neo-antigens restricted to the tumor, the majority of antibodies in cancer patients are directed to self-antigens and are therefore autoantibodies (Bei et al., 2009). Breakthrough of tolerance and elevated levels of autoantibodies to self-antigens are also prominent features of many autoimmune diseases.

Thus, autoantibodies hold the potential to serve as biomarkers of a sustained humoral anti-tumor response and irAE in cancer patients treated with immunotherapeutic approaches.

Compared to biomarker strategies involving the identification of TAA-specific T-cells, the identification of autoantibodies can be performed using modern multiplex high-throughput screening approaches using minimal amounts of serum (Budde et al., 2016).

### BRIEF DESCRIPTION OF DRAWINGS

FIG. 1 depicts a design of the cancer screen. KEGG Pathway Analysis ((Kyoto Encyclopedia of Genes and Genomes) of human (has) proteins and antigens are included in the cancer autoantibody screen. Proteins were selected to represent the following three categories: Tumor and autoimmunity signaling pathways, Immune-related pathways and proteins or genes overexpressed in different cancer types. The number of proteins per category is indicated at the x-axis.

FIG. 2 depicts Box-and-Whisker Plots of four autoantibodies in prostate cancer patients (PCa) and healthy controls (HC). Box-and-Whisker Plots of IgG autoantibody reactivities are shown against CDKN1A, MYLK3 and VASP in serum samples of prostate cancer patients PCa) and healthy controls. A mix of SIPA1 and MCM2 were coupled to the same Luminex bead region. Numbers at the y-axis indicate the Luminex Median Fluorescence Intensity values (MFI).

FIG. 3 depicts a Partial Least Squares (PLS) regression analysis of the autoantibody reactivity in baseline and post-treatment serum samples treated with PROSTVAC. The Partial Least Squares (PLS) Biplot is of Component 5 and 6 of antigens and autoantibodies induced by PROSTVAC treatment (“Study.Day” and pre_post_treatment.post”. The biplot of components 5 versus 6 shows the regression relationship between clinical and demographic predictors shown as vectors in the graph and all autoantibody reactivities. The following predictors were used in the analysis: Age of donor (“age.of.donor”), overall survival (“overall.survival”), time on study as a measure of progression free survival or time to progression (“time.on.study”), sample collected at study day T0, T1, T2 (“study.day”), autoantibodies measure in baseline samples (“pre_post_treatment.pre”) and post-treatment samples T1 and T2 (“pre_post_treatment.post”). In this projection, antigens, which are further away from the origin and located in the vicinity of the vector (“pre_post_treatment.post”) induce an antibody response following PROSTVAC treatment.

FIG. 4 illustrates antigens and autoantibodies correlating with progression-free survival (PFS) in PROSTVAC treated patients. FIG. 4 depicts scatter plots showing examples of autoantibodies correlating with the time patients remained in the study given in days (“time.on.study.days”). This corresponds to the time until progression was observed, which is the time to progression or progression-free survival. FIG. 4 shows autoantibodies reactive with LGALS3BP, SP100, PKN1 and CREM. The y-axis shows the log 2 MFI value of autoantibody reactivity. The Pearson's correlation coefficient and p-value is provided for each autoantibody and shown on top of the graphs.

FIG. 5 depicts scatter plots showing examples of autoantibodies correlating with overall survival (OS) in days. (“Overall.Survival.Days” of PROSTVAC treated patients who remained in the study is given in days (“time.on.study.days”). Antigens and autoantibodies correlate with overall survival (OS) in PROSTVAC treated patients. FIG. 5 shows two autoantibodies reactive with USP33 and TNIP2 with positive correlation to OS. Autoantibodies reactive with MAZ and NOVA2 are negatively correlated with OS and higher levels predict poor OS. The y-axis shows the log 2 MFI value of autoantibody reactivity. The Pearson's correlation coefficient and p-value is provided for each autoantibody and shown on top of the graphs.

FIG. 6 shows a Partial Least Squares (PLS) regression analysis of the autoantibody reactivity in baseline and post-treatment serum samples treated with PROSTVAC plus ipilimumab. Partial Least Squares (PLS) Biplot shows Component 5 and 6 of antigens and autoantibodies induced by PROSTVAC plus ipilumumab treatment (“Study.Day” and pre_post_treatment.post”). The biplot of components 5 versus 6 shows the regression relationship between clinical and demographic predictors shown as vectors in the graph and all autoantibody reactivities. The following predictors were used in the analysis: Age of donor (“age.of.donor”), overall survival (“overall.survival”), time on study (“time.on.study”), sample collected at study day T0, T1, T2 (“study.day”), overall survival (OS) (“best.response”), immune related adverse events (irAE, “Codierung.iRAEs.R17), autoantibodies measured in baseline samples (“pre_post_treatment.pre”) and post-treatment samples T1 and T2 (“pre_post_treatment.post”). In this projection, antigens, which are further away from the origin and located in the vicinity of the vector (“pre_post_treatment.post”) induce an antibody response following PROSTVAC plus ipilimumab treatment.

FIG. 7 illustrates antigens and autoantibodies correlating with OS-Halabi (“best response”) in PROSTVAC plus ipilumumab treated patients. Antigens and autoantibodies correlate with OS-Halabi (“best response”) in PROSTVAC plus ipilumumab treated patients. The scatter plots show examples of autoantibodies correlating with the predicted median OS by the Halabi nomogram (OS-Halabi, “Best.Response”). FIG. 7 shows that autoantibodies reactive with A1BG and ZNF574 are positively correlated to OS-Halabi. Autoantibodies reactive with MAGEA8 and HMMR show negative correlation with OS-Halabi. The y-axis shows the log 2 MFI value of autoantibody reactivity. The Pearson's correlation coefficient and p-value is provided for each autoantibody and shown on top of the graphs.

FIG. 8 illustrates scatter plots showing examples of autoantibodies correlating with overall survival (OS) in days (“Overall.Survival.Days”) of PROSTVAC treated patients. Antigens and autoantibodies correlate with overall survival in days (OS) in PROSTVAC plus ipilumumab treated patients. FIG. 8 shows two autoantibodies reactive with SNRNP70 and RELB with positive correlation to OS. Autoantibodies reactive with HMMR and CREBBP are negatively correlated with OS and higher levels predict poor OS. The y-axis shows the log 2 MFI value of autoantibody reactivity. The Pearson's correlation coefficient and p-value is provided for each autoantibody and shown on top of the graphs.

FIG. 9 depicts a Box-and-Whisker plot of anti-IDO1 antibodies measured in pre-treatment T0 (“pre”) and post-treatment T1 and T2 (“post”) samples. Anti-IDO1 antibodies predict overall survival (OS) in pre-treatment (“pre”)and post-treatment (“post”) samples of prostate cancer patients: Combined analysis of PROSTVAC and PROSTVAC plus ipilimumab. Patient samples were divided into four groups based on their overall survival in month. Anti-IDO1 antibodies predict overall survival (OS) in pre-treatment (“pre”) and are elevated in post-treatment (“post”) samples of prostate cancer patients. FIG. 9 shows the combined analysis of samples from two studies, PROSTVAC and PROSTVAC plus ipilimumab.

FIG. 10 illustrates Box-and-Whisker plots showing two autoantibodies against IRAK4 and RBMS1_c, which show higher levels in cancer patients that develop irAEs following treatment with PROSTVAC plus ipilimumab. Antigens and autoantibodies associated with irAE in PROSTVAC plus ipilumumab treated patients. The test antigen RBMS1_c is an enzymatically modified recombinant protein, in which the amino acid arginine is converted into the amino acid citrulline by a deimination or citrullination reaction. Citrullinated proteins and peptides are well-known antigens of the autoimmune disease rheumatoid arthritis.

### SUMMARY OF THE INVENTION

In one aspect is provided a method of identifying a tumor-associated antigen (TAA) for prostate cancer. A group of patients with prostate cancer is selected. Also, a group of patients who are healthy are selected. A sample from at least one patient in the group with prostate cancer is assayed for the level of an autoantibody to an antigen. The level of the autoantibody to an antigen in the group of patients with prostate cancer is compared to the level of the autoantibody in the group of healthy patients. The antigen is determined to be a TAA for prostate cancer if the level of the autoantibody to the antigen is statistically different between the group of patients with prostate cancer versus the group of healthy patients.

In another aspect is provided a method of identifying a TAA as a marker for prostate cancer vaccination response. A group of patients with prostate cancer who have been vaccinated with a vaccine effective to induce an immune response against a prostate cancer antigen is selected. Also, a group of patients with prostate cancer who have not been vaccinated with the vaccine is selected. A sample from at least one patient in the group with prostate cancer is assayed for the level of an autoantibody to an antigen. The level of the autoantibody to an antigen in the group of patients with prostate cancer who have been vaccinated is compared to the level of the autoantibody in the group of patients with prostate cancer who have not been vaccinated. The antigen is determined to be a TAA for prostate cancer if the level of the autoantibody to the antigen is statistically different between the group of patients who have been vaccinated and the group of patients who have not been vaccinated.

In another aspect is provided a method of identifying and treating a prostate cancer patient with PROSTVAC therapy or for vaccination with a prostate antigen. The level of one or more antigens encoded by a gene listed in Table 4 having a positive value for r_in_PROSTVAC Progression-free survival is determined in a sample from the prostate cancer patient who has undergone PROSTVAC therapy. The level of the same one or more antigens in a sample from a prostate cancer patient, or a group of prostate cancer patients, who have not undergone PROSTVAC therapy. The levels of the one or more antigens in the patient who has undergone PROSTVAC therapy are compared with the corresponding levels of the patient or group of patients who have not undergone PROSTVAC therapy. If the level of the one or more antigens in the patient (encoded by a gene listed in Table 4 having a positive value for r_in_PROSTVAC Progression-free survival) is greater than the average level of the one or more antigens in the group of patients with prostate cancer, then PROSTVAC therapy, Ipilimumab, and/or the vaccination with a prostate antigen is administered to the patient.

Additional aspects and embodiments are described below in the Detailed Description.

### DETAILED DESCRIPTION OF THE INVENTION

In one aspect is provided a method of identifying a tumor-associated antigen (TAA) for prostate cancer. A group of patients with prostate cancer is selected. Also, a group of patients who are healthy are selected. A sample from at least one patient in the group with prostate cancer is assayed for the level of an autoantibody to an antigen. The level of the autoantibody to an antigen in the group of patients with prostate cancer is compared to the level of the autoantibody in the group of healthy patients. The antigen is determined to be a TAA for prostate cancer if the level of the autoantibody to the antigen is statistically different between the group of patients with prostate cancer versus the group of healthy patients.

Within the scope of this invention, the term “patient” is understood to mean any test subject (human or mammal), with the provision that the test subject is tested for prostate cancer.

Autoantibodies can be formed by a patient before prostate cancer progresses or otherwise shows symptoms. Early detection, diagnosis and also prognosis and (preventative) treatment would therefore be possible years before the visible onset of progression. Devices and means (arrangement, array, protein array, diagnostic tool, test kit) and methods described herein can enable a very early intervention compared with known methods, which considerably improves the prognosis and survival rates. Since the prostate cancer-associated autoantibody profiles change during the establishment and treatment/therapy of prostate cancer, the invention also enables the detection and the monitoring of prostate cancer at any stage of development and treatment and also monitoring within the scope of aftercare in the case of prostate cancer. The means according to the invention also allow easy handling at home by the patient himself and cost-effective routine precautionary measures for early detection and also aftercare.

Different patients may have different prostate cancer-associated autoantibody profiles, for example different cohorts or population groups differ from one another. Here, each patient may form one or more different prostate cancer-associated autoantibodies during the course of the development of prostate cancer and the progression of the disease of prostate cancer, that is to say also different autoantibody profiles. In addition, the composition and/or the quantity of the formed prostate cancer-associated autoantibodies may change during the course of the prostate cancer development and progression of the disease, such that a quantitative evaluation is necessary. The therapy/treatment of prostate cancer also leads to changes in the composition and/or the quantity of prostate cancer-associated autoantibodies. The large selection of prostate cancer-associated marker sequences according to the invention allows the individual compilation of prostate cancer-specific marker sequences in an arrangement for individual patients, groups of patients, certain cohorts, population groups, and the like. In an individual case, the use of a prostate cancer-specific marker sequence may therefore be sufficient, whereas in other cases at least two or more prostate cancer-specific marker sequences have to be used together or in combination in order to produce a meaningful autoantibody profile.

Compared with other biomarkers, the detection of prostate cancer-associated autoantibodies for example in the serum/plasma has the advantage of high stability and storage capability and good detectability. The presence of autoantibodies also is not subject to a circadian rhythm, and therefore the sampling is independent of the time of day, food intake and the like.

In addition, the prostate cancer-associated autoantibodies can be detected with the aid of the corresponding antigens/autoantigens in known assays, such as ELISA or Western Blot, and the results can be checked for this.

In some embodiments, the antigen is an antigen encoded by a gene listed in Table 1. In some embodiments, the TAA is encoded by a gene listed in Table 2.

Various ways of performing the assay can be undertaken. A portion of serum from the patient with prostate cancer is contacted with a sample of an antigen. The antigen may be immobilized onto a solid support, in particular a filter, a membrane, a bead or small plate or bead, for example a magnetic or fluorophore-labelled bead, a silicon wafer, glass, metal, plastic, a chip, a mass spectrometry target or a matrix. A microsphere as a solid support may also be used. Multiple antigens may be coupled to multiple different solid supports and then arranged on an array.

The array may be in the form of a “protein array”, which in the sense of this invention is the systematic arrangement of prostate cancer-specific marker sequences on a solid support, wherein the prostate cancer-specific marker sequences are proteins or peptides or parts thereof, and wherein the support is preferably a solid support.

The sample comprising any of the TAAs, autoantigens, autoantibodies, are part of, found in, or otherwise present in, a bodily fluid. The bodily fluid may be blood, whole blood, blood plasma, blood serum, patient serum, urine, cerebrospinal fluid, synovial fluid or a tissue sample, for example from tumour tissue from the patient. These bodily fluids and tissue samples can be used for early detection, diagnosis, prognosis, therapy control and aftercare.

The level of a TAA, autoantibody or antigen is assayed by measuring the degree of binding between a sample and the antigen. Binding according to the invention, binding success, interactions, for example protein-protein interactions (for example protein to prostate cancer-specific marker sequence, such as antigen/antibody) or corresponding “means for detecting the binding success” can be visualised for example by means of fluorescence labelling, biotinylation, radio-isotope labelling or colloid gold or latex particle labelling in the conventional manner. Bound antibodies are detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc. and the corresponding colorimetric, fluorescent or chemiluminescent substrates. A readout is performed, for example, by means of a microarray laser scanner, a CCD camera or visually.

Comparisons may be performed by any number of statistical analyses, such as those described in Example 5 herein.

In another aspect is provided a method of identifying a TAA as a marker for prostate cancer vaccination response. A group of patients with prostate cancer who have been vaccinated with a vaccine effective to induce an immune response against a prostate cancer antigen is selected. Also, a group of patients with prostate cancer who have not been vaccinated with the vaccine is selected. A sample from at least one patient in the group with prostate cancer is assayed for the level of an autoantibody to an antigen. The level of the autoantibody to an antigen in the group of patients with prostate cancer who have been vaccinated is compared to the level of the autoantibody in the group of patients with prostate cancer who have not been vaccinated. The antigen is determined to be a TAA for prostate cancer if the level of the autoantibody to the antigen is statistically different between the group of patients who have been vaccinated and the group of patients who have not been vaccinated.

Another aspect provides a method of identifying and treating a prostate cancer patient with PROSTVAC therapy or for vaccination with a prostate antigen. The level of one or more antigens encoded by a gene listed in Table 4 having a positive value for r_in_PROSTVAC Progression-free survival is determined in a prostate cancer patient. The level of the one or more antigens in the prostate cancer patient is compared with an average level of the one or more antigens for a group of patients with prostate cancer. PROSTVAC therapy, Ipilimumab, and/or vaccination with a prostate antigen is administered if the level of the one or more antigens in the patient is greater than the average level of the one or more antigens in the group of patients with prostate cancer.

Any number of antigens may be tested, such as 2, 3, 4, 5, 6, 7, 8, 9, 10, 11, 12, 13, 14, 15, 16, 17, 18, 19, 20.

In some embodiments, the patient further has a reduced level of one or more antigens encoded by a gene listed in Table 4 having a negative value for r_in_PROSTVAC Progression-free survival as compared to the level in the group of patients with prostate cancer.

PROSTVAC is under development by Bavarian Nordic as a vaccine to be administered to prevent spread of metastatic prostate cancer. PROSTVAC may be helpful to treat men who have symptomatic or minimally symptomatic metastatic castration-resistant prostate cancer (mCRPC). PROSTVAC is a vaccine targeting PSA and is administered by a proprietary prime-boost method. PROSTVAC may be administered subcutaneously. Without wishing to be bound by theory, PROSTVAC may induce a direct immune response that attacks PSA-bearing metastatic prostate cancer cells.

Another aspect provides a method of identifying and treating a prostate cancer patient with PROSTVAC therapy or for vaccination with a prostate antigen. The level of one or more antigens encoded by a gene listed in Table 4 having a negative value for r_in_PROSTVAC Progression-free survival is determined in a prostate cancer patient. The level of the one or more antigens in the prostate cancer patient is compared with an average level of the one or more antigens for a group of patients with prostate cancer. PROSTVAC therapy, Ipilimumab, and/or the vaccination with a prostate antigen is administered if the level of the one or more antigens in the patient is less than the average level of the one or more antigens in the group of patients with prostate cancer.

Another aspect provides a method of monitoring the effectiveness of therapy in a prostate cancer patient previously treated with PROSTVAC vaccination or prostate antigen vaccination. The level of one or more antigens encoded by a gene listed in Table 4 having a negative value for r_in_PROSTVAC Progression-free survival is determined by assaying a sample from a prostate cancer patient. The level of the one or more antigens from the sample of the prostate cancer patient is compared with an average level of the one or more antigens for a group of patients with prostate cancer. A determination that PROSTVAC therapy is effective is made if the level of the one or more antigens in the patient is less than the average level of the one or more antigens in the group of patients with prostate cancer.

Another aspect provides a method of monitoring the effectiveness of therapy in a prostate cancer patient previously treated with PROSTVAC vaccination or prostate antigen vaccination. The level of one or more antigens encoded by a gene listed in Table 4 having a positive value for r_in_PROSTVAC Progression-free survival is determined by assaying the level of one or more antigens in a sample from a prostate cancer patient. The level of the one or more antigens from the sample is compared with an average level of the one or more antigens for a group of patients with prostate cancer. A determination is made that the therapy is effective if the level of the one or more antigens in the patient is greater than the average level of the one or more antigens in the group of patients with prostate cancer.

The therapy may include one or more of Ipilimumab administration, prostate antigen vaccination, and PROSTVAC therapy.

In another aspect is provided a method of identifying and treating a prostate cancer patient previously treated with PROSTVAC vaccination or prostate antigen vaccination. The level of one or more antigens encoded by a gene listed in Table 4 having a positive value for r_in_PROSTVAC Progression-free survival is determined by assaying the level of one or more antigens in a sample from a prostate cancer patient. The level of the one or more antigens is compared with an average level of the one or more antigens for a group of patients with prostate cancer. The therapy or the vaccination with a prostate antigen is administered if the level of the one or more antigens in the patient is greater than the average level of the one or more antigens in the group of patients with prostate cancer

In some embodiments, the administered therapy comprises one or more of Ipilimumab administration, prostate antigen vaccination, and PROSTVAC therapy.

In another aspect is provided a method of identifying and treating a prostate cancer patient previously treated with PROSTVAC vaccination or prostate antigen vaccination. The level of one or more antigens encoded by a gene listed in Table 4 having a negative value for r_in_PROSTVAC Progression-free survival is determined by assaying the level of one or more antigens in a sample from a prostate cancer patient. The level of the one or more antigens from the prostate cancer patient is compared with an average level of the one or more antigens for a group of patients with prostate cancer. Therapy is administered if the level of the one or more antigens in the patient is less than the average level of the one or more antigens in the group of patients with prostate cancer.

In some embodiments, the therapy comprises one or more of Ipilimumab administration, prostate antigen vaccination, and PROSTVAC therapy.

In some embodiments, the patient also has an increased level of one or more antigens encoded by a gene listed in Table 4 having a positive value for r_in_PROSTVAC Progression-free survival as compared to the level in the group of patients with prostate cancer.

In another aspect is provided a method of monitoring the effectiveness of PROSTVAC therapy in a prostate cancer patient previously treated with PROSTVAC therapy or vaccination with a prostate antigen. The level of one or more antigens encoded by a gene listed in Table 4 having a negative value for r_in_PROSTVAC Progression-free survival is determined by assaying the level of one or more antigens in a sample from a prostate cancer patient. The level of the one or more antigens from the prostate cancer patient is compared with an average level of the one or more antigens for a group of patients with prostate cancer. A determination is made that the PROSTVAC therapy is effective if the level of the one or more antigens in the patient is less than the average level of the one or more antigens in the group of patients with prostate cancer.

In another aspect is provided a method of monitoring the effectiveness of PROSTVAC therapy in a prostate cancer patient previously treated with PROSTVAC therapy or vaccination with a prostate antigen. The level of one or more antigens encoded by a gene listed in Table 4 having a positive value for r_in_PROSTVAC Progression-free survival is determined by assaying the level of one or more antigens in a sample from a prostate cancer patient. The level of the one or more antigens in the prostate cancer patient is compared with an average level of the one or more antigens for a group of patients with prostate cancer. A determination is made that the PROSTVAC therapy is effective if the level of the one or more antigens in the patient is greater than the average level of the one or more antigens in the group of patients with prostate cancer.

In another aspect is provided a method of assessing overall survival of a patient who has been treated with PROSTVAC. The level of one or more antigens encoded by a gene listed in Table 5 having a positive value for r_in_Prostvac Overall Survival is determined by assaying the level of one or more antigens in a sample from a prostate cancer patient. The level of the one or more antigens is compared with an average level of the one or more antigens for a group of patients with prostate cancer.

In another aspect is provided a method of monitoring the effectiveness of combined PROSTVAC with Ipilimumab therapy in a prostate cancer patient previously treated with combined PROSTVAC with Ipilimumab therapy. The level of one or more antigens encoded by a gene listed in Table 6 having a positive value for r-value Study.Day is determined by assaying the level of one or more antigens in a sample from a prostate cancer patient. The level of the one or more antigens is compared with an average level of the one or more antigens for a group of patients with prostate cancer. The combined PROSTVAC with Ipilimumab therapy is determined to be effective if the level of the one or more antigens in the patient is greater than the average level of the one or more antigens in the group of patients with prostate cancer.

In yet another aspect is provided a method of monitoring the effectiveness of combined PROSTVAC with Ipilimumab therapy in a prostate cancer patient previously treated with combined PROSTVAC with Ipilimumab therapy. The level of one or more antigens encoded by a gene listed in Table 7 having a positive value for r_in_prostvac_ipi_Best.Response is determined by assaying the level of one or more antigens in a sample from a prostate cancer patient. The level of the one or more antigens is compared with an average level of the one or more antigens for a group of patients with prostate cancer. The combined PROSTVAC with Ipilimumab therapy is determined effective if the level of the one or more antigens in the patient is greater than the average level of the one or more antigens in the group of patients with prostate cancer.

In another aspect is provided a method of assessing overall survival of a patient who has been treated with PROSTVAC and Ipilimumab. The level of one or more antigens encoded by a gene listed in Table 8 having a positive value for r_in_prostvac_ipi_Overall.Survival is determined by assaying the level of one or more antigens in a sample from a prostate cancer patient. The level of the one or more antigens is compared with an average level of the one or more antigens for a group of patients with prostate cancer.

In another aspect is provided a method of monitoring for immune-related adverse events arising from combined PROSTVAC with Ipilimumab therapy in a prostate cancer patient previously treated with combined PROSTVAC with Ipilimumab therapy. The level of one or more antigens encoded by a gene listed in Table 9 having a positive value for Pearson'r is determined by assaying the level of one or more antigens in a sample from a prostate cancer patient. The level of the one or more antigens is compared with an average level of the one or more antigens for a group of patients with prostate cancer. A determination that there is risk for an immune-related adverse event arising from combined PROSTVAC with Ipilimumab therapy is made if the level of the one or more antigens in the patient is greater than the average level of the one or more antigens in the group of patients with prostate cancer.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

### EXAMPLE 1

### Production of Recombinant Autoantigens

Recombinant antigens were produced in Escherichia coli. Five cDNA libraries originating from different human tissues (fetal brain, colon, lung, liver, CD4 induced and non-induced T cells) were used for the recombinant production of human antigens. All of these cDNA libraries were oligo(dT)-primed, containing the coding region for an N-terminally located hexa-histidine-tag and were under transcriptional control of the lactose inducible promoter from E. coli]. Sequence integrity of the cDNA libraries was confirmed by 5′ DNA sequencing. Additionally, expression clones representing the full-length sequence derived from the human ORFeome collection were included. Individual antigens were designed in silico, synthesized chemically (Life Technologies, Carlsbad, USA) and cloned into the expression vector pQE30-NST fused to the coding region for the N-terminal-located His6-tag. Of the antigens. Recombinant gene expression was performed in E. coli SCS1 cells carrying plasmid pSE111 for improved expression of human genes. Cells were cultivated in 200 ml auto-induction medium (Overnight Express auto-induction medium, Merck, Darmstadt, Germany) overnight and harvested by centrifugation. Bacterial pellets were lysed by resuspension in 15 ml lysis buffer (6 M guanidinium-HCl, 0.1 M NaH2PO4, 0.01 M Tris-HCl, pH 8.0).

Soluble proteins were affinity-purified after binding to Protino® Ni-IDA 1000 Funnel Column (Macherey-Nagel, Duren, Germany). Columns were washed with 8 ml washing buffer (8 M urea, 0.1 M NaH2PO4, 0.01 M Tris-HCl, pH 6.3). Proteins were eluted in 3 ml elution buffer (6 M urea, 0.1 M NaH2PO4, 0.01 M Tris-HCl, 0.5% (w/v) trehalose pH 4.5). Each protein preparation was transferred into 2D-barcoded tubes, lyophilized and stored at −20° C.

### EXAMPLE 2

### Selection of Antigens and Design of the Cancer Screen

Candidate antigens were selected for this cancer screen to cover immune-related processes and autoimmune disease antigens, cancer signaling processes, and antigens preferentially expressed in different cancer types. In total, 842 potential antigens were selected.

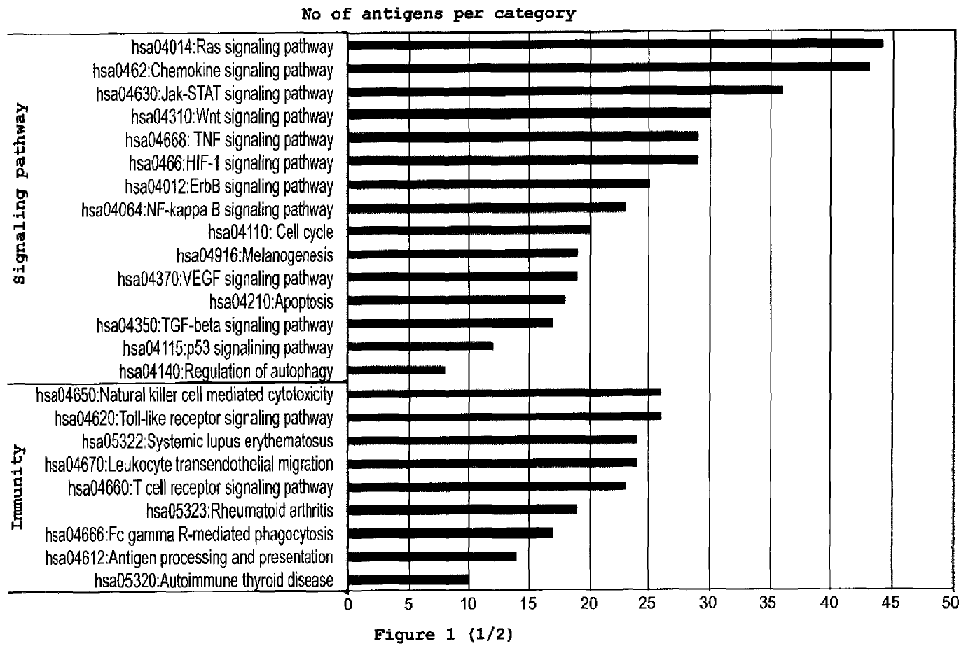

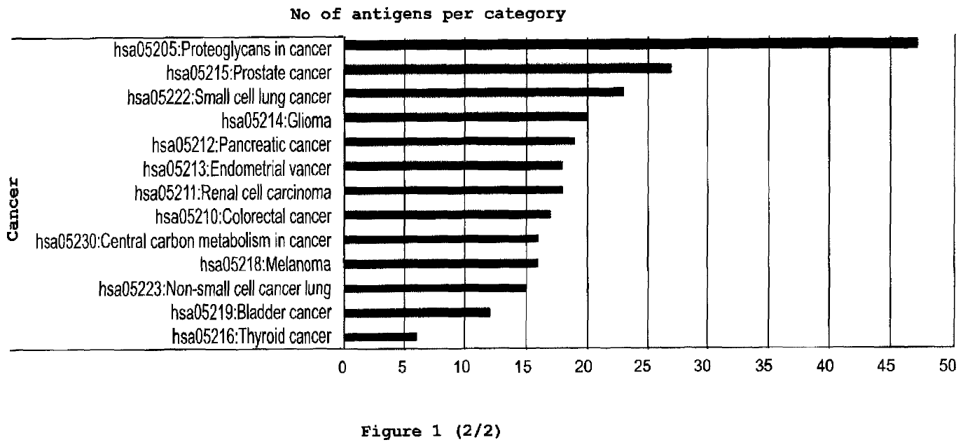

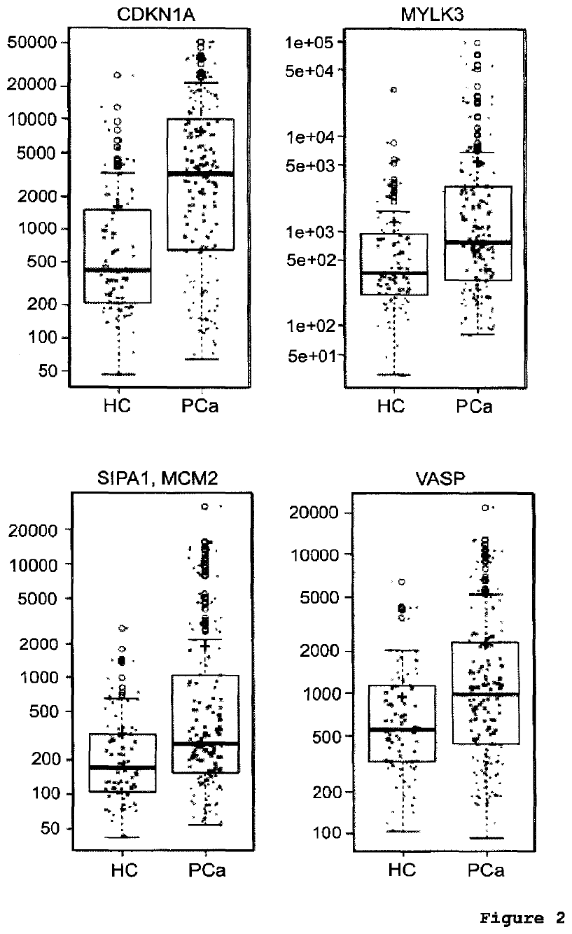

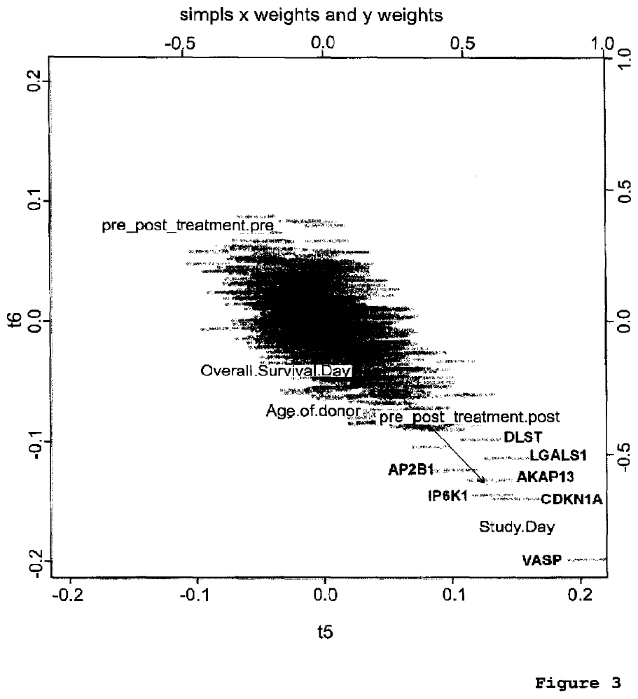

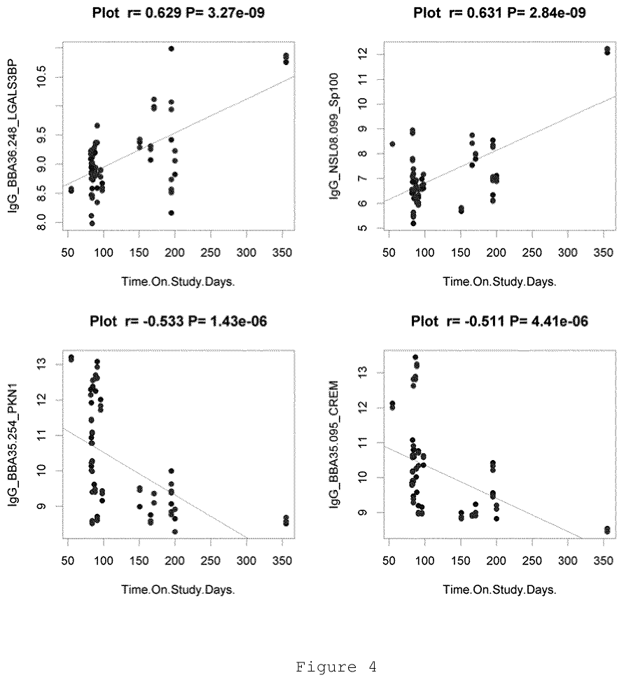

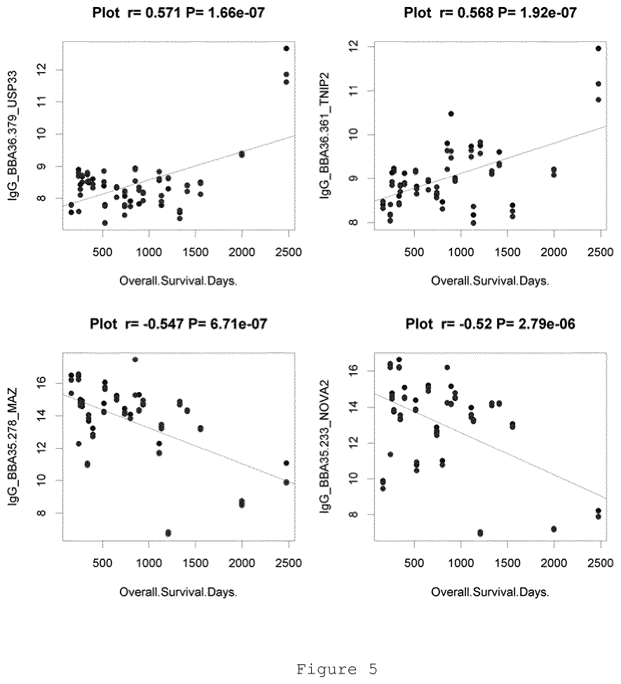

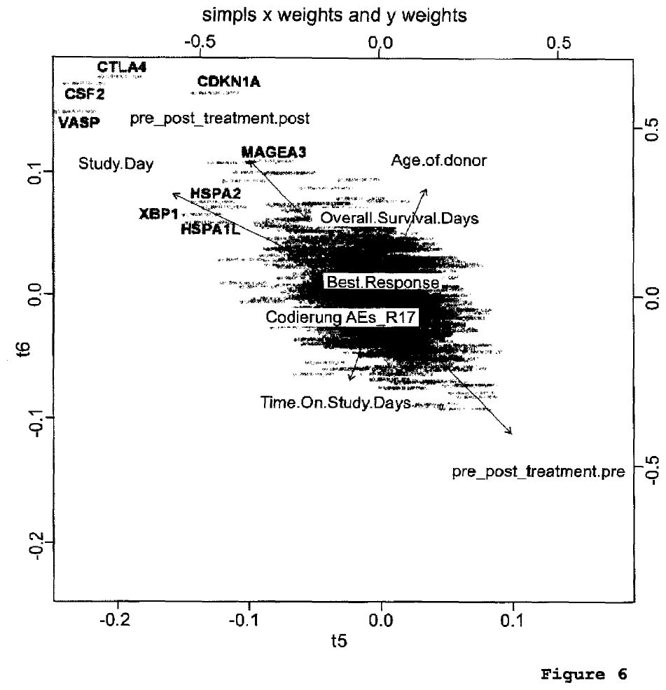

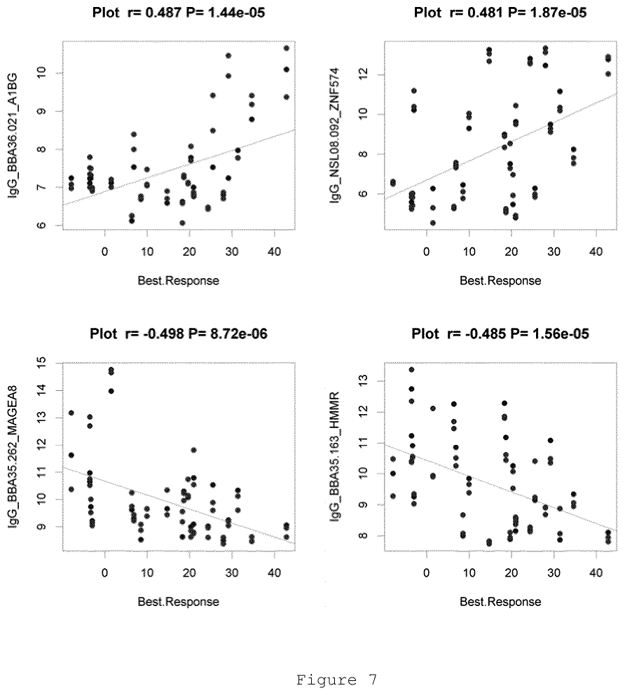

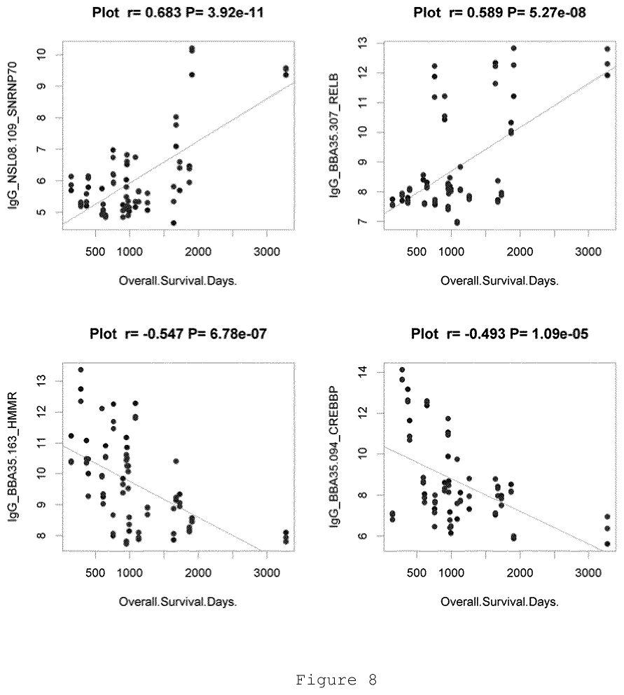

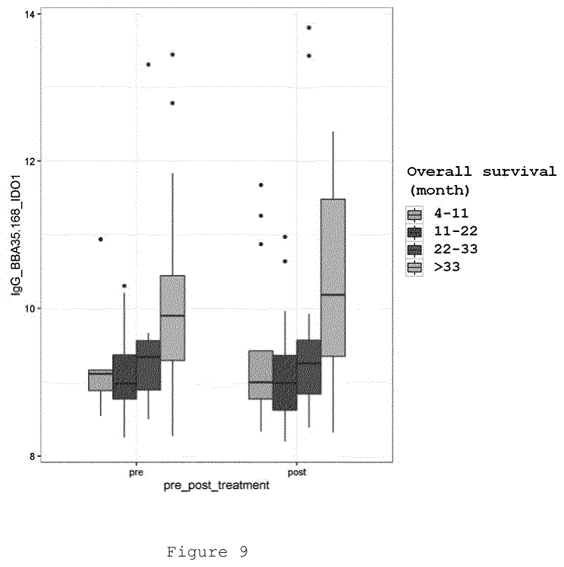

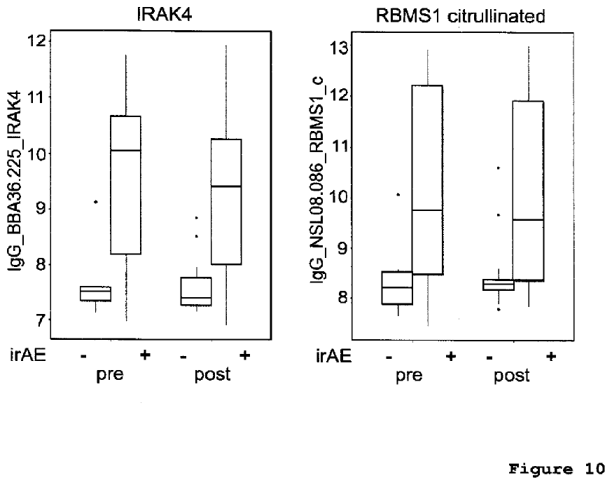

FIG. 1 shows the number of antigens per category.

### EXAMPLE 3

### Coupling of Antigens to Beads

For the production of bead-based arrays (BBA), the proteins were coupled to magnetic carboxylated color-coded beads (MagPlex™ microspheres, Luminex Corporation, Austin, Tex., USA). The manufacturer's protocol for coupling proteins to MagPlexm microspheres was adapted to use liquid handling systems. A semi-automated coupling procedure of one BBA encompassed 384 single, separate coupling reactions, which were carried out in four 96-well plates. For each single coupling reaction, up to 12.5 μg antigen and 8.8×105 MagPlex™ beads of one color region (ID) were used. All liquid handling steps were carried out by either an eight-channel pipetting system (Starlet, Hamilton Robotics, Bonaduz, Switzerland) or a 96-channel pipetting system (Evo Freedom 150, Tecan, Mannderdorf, Switzerland). For semi-automated coupling, antigens were dissolved in H2O, and aliquots of 60 microliters were transferred from 2D barcode tubes to 96-well plates. MagPlex™ microspheres were homogeneously resuspended and each bead ID was transferred in one well of a 96-well plate. The 96-well plates containing the microspheres were placed on a magnetic separator (LifeSep™, Dexter Magnetic Technologies Inc., Elk Grove Village, USA) to sediment the beads for washing steps and on a microtiter plate shaker (MTS2/4, IKA) to facilitate permanent mixing for incubation steps.

For coupling, the microspheres were washed three times with activation buffer (100 mM NaH2PO4, pH 6.2) and resuspended in 120 μl activation buffer. To obtain reactive sulfo-NHS-ester intermediates, 15 μl 1-ethly-3-(3-dimethlyaminopropyl) carbodiimide (50 mg/ml) and 15 μl N-hydroxy-succinimide (50 mg/ml) were applied to microspheres. After 20 minutes incubation (900 rpm, room temperature (RT)) the microspheres were washed three times with coupling buffer (50 mM MES, pH 5.0) and resuspended in 65 μl coupling buffer. Immediately, 60 μl antigen solution was added to reactive microspheres and coupling took place over 120 minutes under permanent mixing (900 rpm, RT). After three wash cycles using washing buffer (PBS, 0.1% Tween20) coupled beads were resuspended in blocking buffer (PBS, 1% BSA, 0.05% ProClin300), incubated for 20 minutes (900 rpm, RT) and then transferred to be maintained at 4-8° C. for 12-72 h.

Finally, a multiplex BBA was produced by pooling 384 antigen-coupled beads.

### EXAMPLE 4

### Incubation of Serum Samples with Antigen-Coupled Beads

Serum samples were transferred to 2D barcode tubes and a 1:100 serum dilution was prepared with assay buffer (PBS, 0.5% BSA, 10% E. coli lysate, 50% Low-Cross buffer (Candor Technologies, Nurnberg, Germany)) in 96-well plates. The serum dilutions were first incubated for 20 minutes to neutralize any human IgG eventually directed against E. coli proteins. The BBA was sonicated for 5 minutes and the bead mix was distributed in 96-well plates. After three wash cycles with washing buffer (PBS, 0.05% Tween20) serum dilutions (50 μl) were added to the bead mix and incubated for 20 h (900 rpm, 4-8° C.). Supernatants were removed from the beads by three wash cycles, and secondary R-phycoerythrin-labeled antibody (5 μg/ml, goat anti-human, Dianova, Hamburg, Germany) was added for a final incubation of 45 minutes (900 rpm, RT). The beads were washed three times with washing buffer (PBS, 0.1% Tween20) and resuspended in 100 μl sheath fluid (Luminex Corporation). Subsequently, beads were analyzed in a FlexMap3D device for fluorescent signal readout (DD gate 7.500-15.000; sample size: 80 μl; 1000 events per bead ID; timeout 60 sec). The binding events were displayed as median fluorescence intensity (MFI). Measurements were disregarded when low numbers of bead events (<30 beads) were counted per bead ID.

### EXAMPLE 5

### Statistical Analysis

Data processing and analysis were performed using the programming language R (http://www.r-project.org/ version 3.3.0), KNIME 3.2 (https://www.knime.org/), DataWarrior (www.openmolecules.org/datawarrior), and tMeV 4.9 (http://www.tm4.org).

To identify autoantibodies that have higher reactivity to the test antigen in a group of patients compared to a control group, the permutation based statistical technique Significance of microarrays in the R-programming language (SAMR) was used (Tusher et al., 2001). The strength of differences between the two test groups is computed as SAMR score_d. A positive fold-change value is indicative of higher autoantibody reactivity in the cancer group compared to healthy control samples. Furthermore, receiver-operating characteristics were calculated to provide area under the curve (AUC) values for each antigen. The ROC curves were generated using the package pROC (Robin et al., 2011).

To identify biomarkers correlating with clinical response, overall survival, study day, or irAE the Pearson's correlation coefficient “r” was calculated.

To explore the data and to identify biomarkers that enable classification and prediction, partial least squares regression (PLS) was applied to the autoantibody (antigens) data set (Palermo et al., 2009). The orthogonal scores algorithm was used to perform the PLS regression using the programming language “R”. Results of PLS modeling were visualized as “biplots” of autoantibodies and demographic, study data and clinical data reflecting the study design. For each antigen coordinate, the distance to the origin indicates the variance in the reduced two-dimensional space. Antigens without variance would lie in the middle of the bi-plot. The identified autoantibody biomarkers were used as landmarks in the graphical representation of the multivariate model.

### EXAMPLE 6

### Identification and Measurement of Antibodies Targeting Tumor-Associated Antigens and Self-Antigens in Prostate Cancer Patients Treated with PROSTVAC

Serum samples from 24 prostate cancer patients treated with PROSTVAC cancer vaccine were tested for the presence of autoantibodies against 842 preselected antigens (Gulley et al., 2014). Samples were collected prior to treatment (T0 samples) and two timepoints during treatment. The T1 corresponds to 90 days (3 month) and the T2 samples corresponds to 180 days (6 month) The PROSTVAC regimen consists of an initial PSA-TRICOM vaccinia-based priming dose, followed by six subsequent PSA-TRICOM boosting doses. These seven injections are given within a 5-month treatment period. To enhance the immune response to weakly immunogenic autoantigens such as PSA, GM-CSF/CSF2 is given at the start of the therapy.

Table 1 includes all identified autoantibody reactivities and antigens.

Markers correlating with different clinical endpoints are extracted and shown in separate tables (T).

TABLE 1
List of all identified antigens

| | Gene | Gene Symbol and | | | | | | | | | |
|---|---|---|---|---|---|---|---|---|---|---|---|
| ID | ID | Antigen Sequence | Gene Name | Table 2 | Table 3 | Table 4 | Table 5 | Table 6 | Table 7 | Table 8 | Table 9 |
| 1 | 3620 | IDO1 (SEQ ID | indoleamine 2,3- | | | | 1 | | 1 | 1 | 1 |
| | | NO: 1) | dioxygenase 1 | | | | | | | | |
| 2 | 1437 | CSF2 (SEQ ID | colony stimulating | 1 | | 1 | 1 | 1 | | | |
| | | NO: 2) | factor 2 | | | | | | | | |
| | | | (granulocyte- | | | | | | | | |
| | | | macrophage) | | | | | | | | |
| 3 | 7408 | VASP (SEQ ID | vasodilator- | 1 | 1 | | | 1 | | | |
| | | NO: 3) | stimulated | | | | | | | | |
| | | | phosphoprotein | | | | | | | | |
| 4 | 1026 | CDKN1A (SEQ ID | cyclin-dependent | 1 | 1 | | | 1 | | | |
| | | NO: 4) | kinase inhibitor 1A | | | | | | | | |
| | | | (p21, Cip1) | | | | | | | | |
| 5 | 1655 | DDX5 (SEQ ID | DEAD (Asp-Glu-Ala- | 1 | | | 1 | | | 1 | |
| | | NO: 5) | Asp) box helicase 5 | | | | | | | | |
| 6 | 6638 | SNRPN (SEQ ID | small nuclear | | | 1 | | | | 1 | 1 |
| | | NO: 6) | ribonucleoprotein | | | | | | | | |
| | | | polypeptide N | | | | | | | | |
| 7 | 11315 | PARK7 (SEQ ID | parkinson protein 7 | 1 | | 1 | | | | | |
| | | NO: 7) | | | | | | | | | |
| 8 | 8503 | PIK3R3 (SEQ ID | phosphoinositide-3- | | | | | | 1 | 1 | 1 |
| | | NO: 8) | kinase, regulatory | | | | | | | | |
| | | | subunit 3 (gamma) | | | | | | | | |
| 9 | 10015 | PDCD6IP (SEQ ID | programmed cell | | | | | 1 | | 1 | 1 |
| | | NO: 9) | death 6 interacting | | | | | | | | |
| | | | protein | | | | | | | | |
| 10 | 3304 | HSPA1B (SEQ ID | heat shock 70 kDa | 1 | | | | | | | 1 |
| | | NO: 10) | protein 1B | | | | | | | | |
| 11 | 2931 | GSK3A (SEQ ID | glycogen synthase | 1 | 1 | | | | 1 | | |
| | | NO: 11) | kinase 3 alpha | | | | | | | | |
| 12 | 1027 | CDKN1B (SEQ ID | cyclin-dependent | 1 | | | | | | | 1 |
| | | NO: 12) | kinase inhibitor 1B | | | | | | | | |
| | | | (p27, Kip1) | | | | | | | | |
| 13 | 26022 | TMEM98 (SEQ ID | transmembrane | | | | | | | 1 | 1 |
| | | NO: 13) | protein 98 | | | | | | | | |
| 14 | 4171 | MCM2 (SEQ ID | minichromosome | 1 | | | | | | 1 | 1 |
| | | NO: 14) | maintenance complex | | | | | | | | |
| | | | component 2 | | | | | | | | |
| 15 | 6494 | SIPA1 (SEQ ID | signal-induced | 1 | | | | | | 1 | 1 |
| | | NO: 15) | proliferation- | | | | | | | | |
| | | | associated 1, | | | | | | | | |
| | | | minichromosome | | | | | | | | |
| | | | maintenance complex | | | | | | | | |
| | | | component 2 | | | | | | | | |
| 16 | 5137 | PDE1C (SEQ ID | phosphodiesterase | | | | 1 | | 1 | 1 | |
| | | NO: 16) | 1C, calmodulin- | | | | | | | | |
| | | | dependent 70 kDa | | | | | | | | |
| 17 | 4843 | NOS2 (SEQ ID | nitric oxide | | | | | | 1 | 1 | 1 |
| | | NO: 17) | synthase 2, | | | | | | | | |
| | | | inducible | | | | | | | | |
| 18 | 7299 | TYR (SEQ ID | tyrosinase | | | | | | 1 | 1 | 1 |
| | | NO: 18) | | | | | | | | | |
| 19 | 9240 | PNMA1 (SEQ ID | paraneoplastic Ma | 1 | | | | | 1 | | |
| | | NO: 19) | antigen 1 | | | | | | | | |
| 20 | 64326 | RFWD2 (SEQ ID | ring finger and WD | 1 | | | | | 1 | | 1 |
| | | NO: 20) | repeat domain 2, E3 | | | | | | | | |
| | | | ubiquitin protein | | | | | | | | |
| | | | ligase | | | | | | | | |
| 21 | 154 | ADRB2 (SEQ ID | adrenoceptor beta | | | | 1 | | | | 1 |
| | | NO: 21) | 2, surface | | | | | | | | |
| 22 | 5971 | RELB (SEQ ID | v-rel avian | | | | | | 1 | 1 | |
| | | NO: 22) | reticuloendotheliosis | | | | | | | | |
| | | | viral oncogene | | | | | | | | |
| | | | homolog B | | | | | | | | |
| 23 | 1493 | CTLA4 (SEQ ID | cytotoxic T- | 1 | | | | 1 | | | |
| | | NO: 23) | lymphocyte | | | | | | | | |
| | | | associated protein 4 | | | | | | | | |
| 24 | 9133 | CCNB2 (SEQ ID | cyclin B2 | | | | 1 | | | 1 | 1 |
| | | NO: 24) | | | | | | | | | |
| 25 | 4107 | MAGEA8 (SEQ ID | melanoma antigen | | | 1 | | | 1 | 1 | |
| | | NO: 25) | family A, 8 | | | | | | | | |
| 26 | 23151 | GRAMD4 (SEQ ID | GRAM domain | | 1 | 1 | | | | | |
| | | NO: 26) | containing 4 | | | | | | | | |
| 27 | 3123 | HLA-DRB1 (SEQ ID | major | | 1 | 1 | | | | | |
| | | NO: 27) | histocompatibility | | | | | | | | |
| | | | complex, class II, | | | | | | | | |
| | | | DR beta 1 | | | | | | | | |
| 28 | 5552 | SRGN (SEQ ID | serglycin | | | 1 | 1 | | | | |
| | | NO: 28) | | | | | | | | | |
| 29 | 6758 | SSX5 (SEQ ID | synovial sarcoma, X | | 1 | 1 | | | | | |
| | | NO: 29) | breakpoint 5 | | | | | | | | |
| 30 | 163 | AP2B1 (SEQ ID | adaptor-related | | 1 | 1 | | | | | |
| | | NO: 30) | protein complex 2, | | | | | | | | |
| | | | beta 1 subunit | | | | | | | | |
| 31 | 7520 | XRCC5 (SEQ ID | X-ray repair | | | 1 | 1 | | | | |
| | | NO: 31) | complementing | | | | | | | | |
| | | | defective repair in | | | | | | | | |
| | | | Chinese hamster | | | | | | | | |
| | | | cells 5 (double- | | | | | | | | |
| | | | strand-break | | | | | | | | |
| | | | rejoining) | | | | | | | | |
| 32 | 3554 | IL1R1 (SEQ ID | interleukin 1 | | | 1 | | | | | 1 |
| | | NO: 32) | receptor, type I | | | | | | | | |
| 33 | 6625 | SNRNP70 (SEQ ID | U1-snRNP 68/70 kDa | | | | | | 1 | 1 | |
| | | NO: 33) | | | | | | | | | |
| 34 | 1743 | DLST (SEQ ID | OGDC-E2 | | 1 | | | | | | |
| | | NO: 34) | | | | | | | | | |
| 35 | 1857 | DVL3 (SEQ ID | dishevelled segment | 1 | | | | | | | |
| | | NO: 35) | polarity protein 3 | | | | | | | | |
| 36 | 3280 | HES1 (SEQ ID | hes family bHLH | | | 1 | | 1 | | | |
| | | NO: 36) | transcription | | | | | | | | |
| | | | factor 1 | | | | | | | | |
| 37 | 5585 | PKN1 (SEQ ID | protein kinase N1 | | | 1 | 1 | | | | |
| | | NO: 37) | | | | | | | | | |
| 38 | 311 | ANXA11 (SEQ ID | annexin A11 | | | 1 | | | | | 1 |
| | | NO: 38) | | | | | | | | | |
| 39 | 3624 | INHBA (SEQ ID | inhibin, beta A | | | 1 | 1 | | | | |
| | | NO: 39) | | | | | | | | | |
| 40 | 208 | AKT2 (SEQ ID | v-akt murine | | 1 | 1 | | | | | |
| | | NO: 40) | thymoma viral | | | | | | | | |
| | | | oncogene homolog 2 | | | | | | | | |
| 41 | 1785 | DNM2 (SEQ ID | dynamin 2 | | | 1 | 1 | | | | |
| | | NO: 41) | | | | | | | | | |
| 42 | 729447 | GAGE2A (SEQ ID | G antigen 2A | | | | | 1 | | | |
| | | NO: 42) | | | | | | | | | |
| 43 | 729408 | GAGE2D (SEQ ID | G antigen 2D | | | | | 1 | | | |
| | | NO: 43) | | | | | | | | | |
| 44 | 26749 | GAGE2E (SEQ ID | G antigen 2E | | | | | 1 | | | |
| | | NO: 44) | | | | | | | | | |
| 45 | 6195 | RPS6KA1 (SEQ ID | ribosomal protein | 1 | 1 | | | | | | |
| | | NO: 45) | S6 kinase, 90 kDa, | | | | | | | | |
| | | | polypeptide 1 | | | | | | | | |
| 46 | 273 | AMPH (SEQ ID | amphiphysin | | | | | | 1 | | 1 |
| | | NO: 46) | | | | | | | | | |
| 47 | 3397 | ID1 (SEQ ID | inhibitor of DNA | | | | | | 1 | 1 | |
| | | NO: 47) | binding 1, dominant | | | | | | | | |
| | | | negative helix- | | | | | | | | |
| | | | loop-helix protein | | | | | | | | |
| 48 | 3956 | LGALS1 (SEQ ID | lectin, | | 1 | | | 1 | | | |
| | | NO: 48) | galactoside- | | | | | | | | |
| | | | binding, soluble, 1 | | | | | | | | |
| 49 | 79155 | TNIP2 (SEQ ID | TNFAIP3 interacting | | | | 1 | | 1 | | |
| | | NO: 49) | protein 2 | | | | | | | | |
| 50 | 3305 | HSPA1L (SEQ ID | heat shock 70 kDa | | 1 | | | 1 | | | |
| | | NO: 50) | protein 1-like | | | | | | | | |
| 51 | 8320 | EOMES (SEQ ID | eomesodermin | 1 | | | | | 1 | | |
| | | NO: 51) | | | | | | | | | |
| 52 | 9173 | IL1RL1 (SEQ ID | interleukin 1 | | | | 1 | | | | 1 |
| | | NO: 52) | receptor-like 1 | | | | | | | | |
| 53 | 6624 | FSCN1 (SEQ ID | fascin actin- | | | | | | | 1 | 1 |
| | | NO: 53) | bundling protein 1 | | | | | | | | |
| 54 | 23646 | PLD3 (SEQ ID | phospholipase D | | | | | | 1 | 1 | |
| | | NO: 54) | family, member 3 | | | | | | | | |
| 55 | 491 | ATP2B2 (SEQ ID | ATPase, Ca++ | | 1 | | | | | 1 | |
| | | NO: 55) | transporting, | | | | | | | | |
| | | | plasma membrane 2 | | | | | | | | |
| 56 | 91807 | MYLK3 (SEQ ID | myosin light chain | 1 | | | | | | | |
| | | NO: 56) | kinase 3 | | | | | | | | |
| 57 | 30850 | CDR2L (SEQ ID | cerebellar | | | | | | 1 | 1 | |
| | | NO: 57) | degeneration- | | | | | | | | |
| | | | related protein 2- | | | | | | | | |
| | | | like | | | | | | | | |
| 58 | 4436 | MSH2 (SEQ ID | mutS homolog 2 | 1 | | | | | 1 | | |
| | | NO: 58) | | | | | | | | | |
| 59 | 1211 | CLTA (SEQ ID | clathrin, light | 1 | | | | | | | 1 |
| | | NO: 59) | chain A | | | | | | | | |
| 60 | 3312 | HSPA8 (SEQ ID | heat shock 70 kDa | 1 | | | | 1 | | | |
| | | NO: 60) | protein 8 | | | | | | | | |
| 61 | 9094 | UNC119 (SEQ ID | unc-119 homolog | | | | | | | | 1 |
| | | NO: 61) | (C. elegans) | | | | | | | | |
| 62 | 891 | CCNB1 (SEQ ID | cyclin B1 | 1 | | | | 1 | | | |
| | | NO: 62) | | | | | | | | | |
| 63 | 30848 | CTAG2 (SEQ ID | cancer/testis | | | | 1 | | 1 | | |
| | | NO: 63) | antigen 2 | | | | | | | | |
| 64 | 30848 | CTAG2 (SEQ ID | cancer/testis | | | | 1 | | 1 | | |
| | | NO: 64) | antigen 2 | | | | | | | | |
| 65 | 1387 | CREBBP (SEQ ID | CREB binding | | | | | | 1 | 1 | |
| | | NO: 65) | protein | | | | | | | | |
| 66 | 64763 | ZNF574 (SEQ ID | zinc finger protein | | | | | | 1 | 1 | |
| | | NO: 66) | 574 | | | | | | | | |
| 67 | 408 | ARRB1 (SEQ ID | arrestin, beta 1 | | | | | | | 1 | |
| | | NO: 67) | | | | | | | | | |
| 68 | 59067 | IL21 (SEQ ID | interleukin 21 | | | | | | 1 | | 1 |
| | | NO: 68) | | | | | | | | | |
| 69 | 2045 | EPHA7 (SEQ ID | EPH receptor A7 | | | | | | 1 | 1 | |
| | | NO: 69) | | | | | | | | | |
| 70 | 10411 | RAPGEF3 (SEQ ID | Rap guanine | 1 | | | 1 | | | | |
| | | NO: 70) | nucleotide exchange | | | | | | | | |
| | | | factor (GEF) 3 | | | | | | | | |
| 71 | 8517 | IKBKG (SEQ ID | inhibitor of kappa | | | | 1 | 1 | | | |
| | | NO: 71) | light polypeptide | | | | | | | | |
| | | | gene enhancer in B- | | | | | | | | |
| | | | cells, kinase gamma | | | | | | | | |
| 72 | 5223 | PGAM1 (SEQ ID | phosphoglycerate | | | | | | 1 | 1 | |
| | | NO: 72) | mutase 1 (brain) | | | | | | | | |
| 73 | 283431 | GAS2L3 (SEQ ID | growth arrest- | | | | | | 1 | 1 | |
| | | NO: 73) | specific 2 like 3 | | | | | | | | |
| 74 | 5937 | RBMS1 (SEQ ID | RNA binding motif, | | | | | | | 1 | 1 |
| | | NO: 74) | single stranded | | | | | | | | |
| | | | interacting protein 1 | | | | | | | | |
| 75 | 6181 | RPLP2 (SEQ ID | ribosomal protein, | | | | | | | 1 | 1 |
| | | NO: 75) | large, P2 | | | | | | | | |
| 76 | 130617 | ZFAND2B (SEQ ID | zinc finger, AN1- | 1 | | | | | | | 1 |
| | | NO: 76) | type domain 2B | | | | | | | | |
| 77 | 6749 | SSRP1 (SEQ ID | structure specific | | | | | | | | |
| | | NO: 77) | recognition protein 1 | | | | | | | | |
| 78 | 841 | CASP8 (SEQ ID | caspase 8, | 1 | | | 1 | | | | |
| | | NO: 78) | apoptosis-related | | | | | | | | |
| | | | cysteine peptidase | | | | | | | | |
| 79 | 843 | CASP10 (SEQ ID | caspase 10, | | | | 1 | | | | |
| | | NO: 79) | apoptosis-related | | | | | | | | |
| | | | cysteine peptidase | | | | | | | | |
| 80 | 3908 | LAMA2 (SEQ ID | laminin, alpha 2 | | | | | | | 1 | 1 |
| | | NO: 80) | | | | | | | | | |
| 81 | 310 | ANXA7 (SEQ ID | annexin A7 | | 1 | | | | | | 1 |
| | | NO: 81) | | | | | | | | | |
| 82 | 1060 | CENPC (SEQ ID | centromere protein | | | | | | 1 | 1 | |
| | | NO: 82) | C | | | | | | | | |
| 83 | 3161 | HMMR (SEQ ID | hyaluronan-mediated | | | | | | 1 | 1 | |
| | | NO: 83) | motility receptor | | | | | | | | |
| | | | (RHAMM) | | | | | | | | |
| 84 | 283748 | PLA2G4D (SEQ ID | phospholipase A2, | | | | | | 1 | 1 | |
| | | NO: 84) | group IVD | | | | | | | | |
| | | | (cytosolic) | | | | | | | | |
| 85 | 8490 | RGS5 (SEQ ID | regulator of G- | | | | 1 | | 1 | | |
| | | NO: 85) | protein signaling 5 | | | | | | | | |
| 86 | 9275 | BCL7B (SEQ ID | B-cell CLL/lymphoma | 1 | | | 1 | | | | |
| | | NO: 86) | 7B | | | | | | | | |
| 87 | 27179 | IL36A (SEQ ID | interleukin 36, | 1 | | | 1 | | | | |
| | | NO: 87) | alpha | | | | | | | | |
| 88 | 5906 | RAP1A (SEQ ID | RAP1A, member of | | | | | | 1 | | |
| | | NO: 88) | RAS oncogene family | | | | | | | | |
| 89 | 1 | A1BG (SEQ ID | alpha-1-B | | | | | | 1 | 1 | |
| | | NO: 89) | glycoprotein | | | | | | | | |
| 90 | 2316 | FLNA (SEQ ID | filamin A, alpha | 1 | | | | | | | |
| | | NO: 90) | | | | | | | | | |
| 91 | 6464 | SHC1 (SEQ ID | SHC (Src homology 2 | | | | | | 1 | 1 | |
| | | NO: 91) | domain containing) | | | | | | | | |
| | | | transforming | | | | | | | | |
| | | | protein 1 | | | | | | | | |
| 92 | 5705 | PSMC5 (SEQ ID | proteasome | | | | | | | | 1 |
| | | NO: 92) | (prosome, | | | | | | | | |
| | | | macropain) 26S | | | | | | | | |
| | | | subunit, ATPase, 5 | | | | | | | | |
| 93 | 4802 | NFYC (SEQ ID | nuclear | | | | | | | | 1 |
| | | NO: 93) | transcription | | | | | | | | |
| | | | factor Y, gamma | | | | | | | | |
| 94 | 10724 | MGEA5 (SEQ ID | meningioma | 1 | | | | | | | |
| | | NO: 94) | expressed antigen 5 | | | | | | | | |
| | | | (hyaluronidase) | | | | | | | | |
| 95 | 10285 | SMNDC1 (SEQ ID | survival motor | | | | | 1 | | | 1 |
| | | NO: 95) | neuron domain | | | | | | | | |
| | | | containing 1 | | | | | | | | |
| 96 | 10000 | AKT3 (SEQ ID | v-akt murine | | | | | | 1 | 1 | |
| | | NO: 96) | thymoma viral | | | | | | | | |
| | | | oncogene homolog 3 | | | | | | | | |
| 97 | 5801 | PTPRR (SEQ ID | protein tyrosine | | | | | | 1 | 1 | |
| | | NO: 97) | phosphatase, | | | | | | | | |
| | | | receptor type, R | | | | | | | | |
| 98 | 2099 | ESR1 (SEQ ID | estrogen receptor 1 | | | | | | 1 | 1 | |
| | | NO: 98) | | | | | | | | | |
| 99 | 4796 | TONSL (SEQ ID | tonsoku-like, DNA | 1 | | | | | | | |
| | | NO: 99) | repair protein | | | | | | | | |
| 100 | 6757 | SSX2 (SEQ ID | synovial sarcoma, X | | | 1 | | | | | |
| | | NO: 100) | breakpoint 2 | | | | | | | | |
| 101 | 2920 | CXCL2 (SEQ ID | chemokine (C-X-C | | | 1 | | | | | |
| | | NO: 101) | motif) ligand 2 | | | | | | | | |
| 102 | 23367 | LARP1 (SEQ ID | La | | | 1 | | | | | |
| | | NO: 102) | ribonucleoprotein | | | | | | | | |
| | | | domain family, | | | | | | | | |
| | | | member 1 | | | | | | | | |
| 103 | 2921 | CXCL3 (SEQ ID | chemokine (C-X-C | | | 1 | | | | | |
| | | NO: 103) | motif) ligand 3 | | | | | | | | |
| 104 | 4110 | MAGEA11 (SEQ ID | melanoma antigen | | | | | | | | |
| | | NO: 104) | family A, 11 | | | | | | | | |
| 105 | 6672 | Sp100 (SEQ ID | Sp100 | | | 1 | | | | | |
| | | NO: 105) | | | | | | | | | |
| 106 | 3959 | LGALS3BP (SEQ ID | lectin, | | | 1 | | | | | |
| | | NO: 106) | galactoside- | | | | | | | | |
| | | | binding, soluble, 3 | | | | | | | | |
| | | | binding protein | | | | | | | | |
| 107 | 1845 | DUSP3 (SEQ ID | dual specificity | | | 1 | | | | | |
| | | NO: 107) | phosphatase 3 | | | | | | | | |
| 108 | 1048 | CEACAM5 (SEQ ID | carcinoembryonic | | | 1 | | | | | |
| | | NO: 108) | antigen-related | | | | | | | | |
| | | | cell adhesion | | | | | | | | |
| | | | molecule 5 | | | | | | | | |
| 109 | 29115 | SAP30BP (SEQ ID | SAP30 binding | | | 1 | | | | | |
| | | NO: 109) | protein | | | | | | | | |
| 110 | 2633 | GBP1 (SEQ ID | guanylate binding | | | 1 | | | | | |
| | | NO: 110) | protein 1, | | | | | | | | |
| | | | interferon- | | | | | | | | |
| | | | inducible | | | | | | | | |
| 111 | 593 | BCKDHA (SEQ ID | branched chain keto | | | 1 | | | | | |
| | | NO: 111) | acid dehydrogenase | | | | | | | | |
| | | | E1, alpha | | | | | | | | |
| | | | polypeptide | | | | | | | | |
| 112 | 2335 | FN1 (SEQ ID | fibronectin 1 | | | 1 | | | | | |
| | | NO: 112) | | | | | | | | | |
| 113 | 3002 | GZMB (SEQ ID | granzyme B | | | 1 | | | | | |
| | | NO: 113) | (granzyme 2, | | | | | | | | |
| | | | cytotoxic T- | | | | | | | | |
| | | | lymphocyte- | | | | | | | | |
| | | | associated serine | | | | | | | | |
| | | | esterase 1) | | | | | | | | |
| 114 | 140462 | ASB9 (SEQ ID | ankyrin repeat and | | | 1 | | | | | |
| | | NO: 114) | SOCS box containing 9 | | | | | | | | |
| 115 | 7157 | TP53 (SEQ ID | tumor protein p53 | | | 1 | | | | | |
| | | NO: 115) | | | | | | | | | |
| 116 | 4001 | LMNB1 (SEQ ID | lamin B1 | | | 1 | | | | | |
| | | NO: 116) | | | | | | | | | |
| 117 | 468 | ATF4 (SEQ ID | activating | | | 1 | | | | | |
| | | NO: 117) | transcription | | | | | | | | |
| | | | factor 4 | | | | | | | | |
| 118 | 2288 | FKBP4 (SEQ ID | FK506 binding | | | 1 | | | | | |
| | | NO: 118) | protein 4, 59 kDa | | | | | | | | |
| 119 | 5533 | PPP3CC (SEQ ID | protein phosphatase | | | 1 | | | | | |
| | | NO: 119) | 3, catalytic | | | | | | | | |
| | | | subunit, gamma | | | | | | | | |
| | | | isozyme | | | | | | | | |
| 120 | 2157 | F8 (SEQ ID | coagulation factor | | | 1 | | | | | |
| | | NO: 120) | VIII, procoagulant | | | | | | | | |
| | | | component | | | | | | | | |
| 121 | 57568 | SIPA1L2 (SEQ ID | signal-induced | | | 1 | | | | | |
| | | NO: 121) | proliferation- | | | | | | | | |
| | | | associated 1 like 2 | | | | | | | | |
| 122 | 23256 | SCFD1 (SEQ ID | sec1 family domain | | | 1 | | | | | |
| | | NO: 122) | containing 1 | | | | | | | | |
| 123 | 55827 | DCAF6 (SEQ ID | DDB1 and CUL4 | | | 1 | | | | | |
| | | NO: 123) | associated factor 6 | | | | | | | | |
| 124 | 60560 | NAA35 (SEQ ID | N(alpha)- | | | | | | | | 1 |
| | | NO: 124) | acetyltransferase | | | | | | | | |
| | | | 35, NatC auxiliary | | | | | | | | |
| | | | subunit | | | | | | | | |
| 125 | 1457 | CSNK2A1 (SEQ ID | casein kinase 2, | | | | | | | | |
| | | NO: 125) | alpha 1 polypeptide | | | | | | | | |
| 126 | 2919 | CXCL1 (SEQ ID | chemokine (C-X-C | | | | | | | 1 | |
| | | NO: 126) | motif) ligand 1 | | | | | | | | |
| | | | (melanoma growth | | | | | | | | |
| | | | stimulating | | | | | | | | |
| | | | activity, alpha) | | | | | | | | |
| 127 | 27332 | ZNF638 (SEQ ID | zinc finger protein | | 1 | | | | | | |
| | | NO: 127) | 638 | | | | | | | | |
| 128 | 5034 | P4HB (SEQ ID | prolyl 4- | | | | | | | | 1 |
| | | NO: 128) | hydroxylase, beta | | | | | | | | |
| | | | polypeptide | | | | | | | | |
| 129 | 8648 | NCOA1 (SEQ ID | nuclear receptor | | | | 1 | | | | |
| | | NO: 129) | coactivator 1 | | | | | | | | |
| 130 | 7157 | TP53 (SEQ ID | tumor protein p53 | | | 1 | | | | | |
| | | NO: 130) | | | | | | | | | |
| 131 | 1487 | CTBP1 (SEQ ID | C-terminal binding | | | | | | | | 1 |
| | | NO: 131) | protein 1 | | | | | | | | |
| 132 | 1639 | DCTN1 (SEQ ID | dynactin 1 | | | | | 1 | | | |
| | | NO: 132) | | | | | | | | | |
| 133 | 5493 | PPL (SEQ ID | periplakin | | 1 | | | | | | |
| | | NO: 133) | | | | | | | | | |
| 134 | 5957 | RCVRN (SEQ ID | recoverin | | | | | 1 | | | |
| | | NO: 134) | | | | | | | | | |
| 135 | 2266 | FGG (SEQ ID | fibrinogen gamma | | | | 1 | | | | |
| | | NO: 135) | chain | | | | | | | | |
| 136 | 10818 | FRS2 (SEQ ID | fibroblast growth | 1 | | | | | | | |
| | | NO: 136) | factor receptor | | | | | | | | |
| | | | substrate 2 | | | | | | | | |
| 137 | 3306 | HSPA2 (SEQ ID | heat shock 70 kDa | | | | | 1 | | | |
| | | NO: 137) | protein 2 | | | | | | | | |
| 138 | 83593 | RASSF5 (SEQ ID | Ras association | | | 1 | | | | | |
| | | NO: 138) | (RalGDS/AF-6) | | | | | | | | |
| | | | domain family | | | | | | | | |
| | | | member 5 | | | | | | | | |
| 139 | 9909 | DENND4B (SEQ ID | DENN/MADD domain | | | 1 | | | | | |
| | | NO: 139) | containing 4B | | | | | | | | |
| 140 | 1390 | CREM (SEQ ID | cAMP responsive | | | 1 | | | | | |
| | | NO: 140) | element modulator | | | | | | | | |
| 141 | 2934 | GSN (SEQ ID | gelsolin | | | | | | | | 1 |
| | | NO: 141) | | | | | | | | | |
| 142 | 5934 | RBL2 (SEQ ID | retinoblastoma-like | | | | | | 1 | | |
| | | NO: 142) | 2 | | | | | | | | |
| 143 | 3611 | ILK (SEQ ID | integrin-linked | | | | | | 1 | | |
| | | NO: 143) | kinase | | | | | | | | |
| 144 | 672 | BRCA1 (SEQ ID | breast cancer 1, | | | | | | | | 1 |
| | | NO: 144) | early onset | | | | | | | | |
| 145 | 3566 | IL4R (SEQ ID | interleukin 4 | | | | 1 | | | | |
| | | NO: 145) | receptor | | | | | | | | |
| 146 | 6374 | CXCL5 (SEQ ID | chemokine (C-X-C | | 1 | | | | | | |
| | | NO: 146) | motif) ligand 5 | | | | | | | | |
| 147 | 30011 | SH3KBP1 (SEQ ID | SH3-domain kinase | | | | | | | | 1 |
| | | NO: 147) | binding protein 1 | | | | | | | | |
| 148 | 26037 | SIPA1L1 (SEQ ID | signal-induced | | | | | | | | 1 |
| | | NO: 148) | proliferation- | | | | | | | | |
| | | | associated 1 like 1 | | | | | | | | |
| 149 | 7791 | ZYX (SEQ ID | zyxin | | | | | | | | |
| | | NO: 149) | | | | | | | | | |
| 150 | 29968 | PSAT1 (SEQ ID | phosphoserine | | | | 1 | | | | |
| | | NO: 150) | aminotransferase 1 | | | | | | | | |
| 151 | 4088 | SMAD3 (SEQ ID | SMAD family member | | | | | | | 1 | |
| | | NO: 151) | 3 | | | | | | | | |
| 152 | 10454 | TAB1 (SEQ ID | TGF-beta activated | 1 | | | | | | | |
| | | NO: 152) | kinase 1/MAP3K7 | | | | | | | | |
| | | | binding protein 1 | | | | | | | | |
| 153 | 65264 | UBE2Z (SEQ ID | ubiquitin- | | | | | | 1 | | |
| | | NO: 153) | conjugating enzyme | | | | | | | | |
| | | | E2Z | | | | | | | | |
| 154 | 8971 | H1FX (SEQ ID | H1 histone family, | | | | 1 | | | | |
| | | NO: 154) | member X | | | | | | | | |
| 155 | 7494 | XBP1 (SEQ ID | X-box binding | | | | | 1 | | | |
| | | NO: 155) | protein 1 | | | | | | | | |
| 156 | 629 | CFB (SEQ ID | complement factor B | | | | 1 | | | | |
| | | NO: 156) | | | | | | | | | |
| 157 | 1287 | COL4A5 (SEQ ID | collagen, type IV, | | | | | | 1 | | |
| | | NO: 157) | alpha 5 | | | | | | | | |
| 158 | 307 | ANXA4 (SEQ ID | annexin A4 | | 1 | | | | | | |
| | | NO: 158) | | | | | | | | | |
| 159 | 23299 | BICD2 (SEQ ID | bicaudal D homolog | | | | | | | | 1 |
| | | NO: 159) | 2 (Drosophila) | | | | | | | | |
| 160 | 10718 | NRG3 (SEQ ID | neuregulin 3 | | | | | | | | 1 |
| | | NO: 160) | | | | | | | | | |
| 161 | 25865 | PRKD2 (SEQ ID | protein kinase D2 | | | | 1 | | | | |
| | | NO: 161) | | | | | | | | | |
| 162 | 10938 | EHD1 (SEQ ID | EH-domain | | | | | | | 1 | |
| | | NO: 162) | containing 1 | | | | | | | | |
| 163 | 1174 | AP1S1 (SEQ ID | adaptor-related | | | | | | | 1 | |
| | | NO: 163) | protein complex 1, | | | | | | | | |
| | | | sigma 1 subunit | | | | | | | | |
| 164 | 23032 | USP33 (SEQ ID | ubiquitin specific | | | | 1 | | | | |
| | | NO: 164) | peptidase 33 | | | | | | | | |
| 165 | 483 | ATP1B3 (SEQ ID | ATPase, Na+/K+ | | 1 | | | | | | |
| | | NO: 165) | transporting, beta | | | | | | | | |
| | | | 3 polypeptide | | | | | | | | |
| 166 | 2547; | XRCC6 (SEQ ID | Ku (p70, p80) | | | | | | | | |
| | 7520 | NO: 166); XRCC5 | | | | | | | | | |
| | | (SEQ ID NO: 167) | | | | | | | | | |
| 167 | 4069 | LYZ (SEQ ID | lysozyme | | | | | | | 1 | |
| | | NO: 168) | | | | | | | | | |
| 168 | 7343 | UBTF (SEQ ID | upstream binding | | | | 1 | | | | |
| | | NO: 169) | transcription | | | | | | | | |
| | | | factor, RNA | | | | | | | | |
| | | | polymerase I | | | | | | | | |
| 169 | 4000 | LMNA (SEQ ID | lamin A/C | | | | | | | 1 | |
| | | NO: 170) | | | | | | | | | |
| 170 | 80184 | CEP290 (SEQ ID | centrosomal protein | | | | | | | | 1 |
| | | NO: 171) | 290 kDa | | | | | | | | |
| 171 | 2870 | GRK6 (SEQ ID | G protein-coupled | | | | | | | 1 | |
| | | NO: 172) | receptor kinase 6 | | | | | | | | |
| 172 | 6434 | TRA2B (SEQ ID | transformer 2 beta | 1 | | | | | | | |
| | | NO: 173) | homolog | | | | | | | | |
| | | | (Drosophila) | | | | | | | | |
| 173 | 30827 | CXXC1 (SEQ ID | CXXC finger protein | | | | | 1 | | | |
| | | NO: 174) | 1 | | | | | | | | |
| 174 | 3146 | HMGB1 (SEQ ID | high mobility group | | | | 1 | | | | |
| | | NO: 175) | box 1 | | | | | | | | |
| 175 | 7167 | TPI1 (SEQ ID | triosephosphate | | | | | 1 | | | |
| | | NO: 176) | isomerase 1 | | | | | | | | |
| 176 | 80184 | CEP290 (SEQ ID | centrosomal protein | | | | | | | 1 | |
| | | NO: 177) | 290 kDa | | | | | | | | |
| 177 | 23396 | PIP5K1C (SEQ ID | phosphatidylinositol- | | | | 1 | | | | |
| | | NO: 178) | 4-phosphate 5- | | | | | | | | |
| | | | kinase, type I, | | | | | | | | |
| | | | gamma | | | | | | | | |
| 178 | 3875 | KRT18 (SEQ ID | keratin 18, type I | | | | | | | | 1 |
| | | NO: 179) | | | | | | | | | |
| 179 | 1938 | EEF2 (SEQ ID | eukaryotic | | | | | | 1 | | |
| | | NO: 180) | translation | | | | | | | | |
| | | | elongation factor 2 | | | | | | | | |
| 180 | 1938 | EEF2 (SEQ ID | eukaryotic | | | | | | 1 | | |
| | | NO: 180) | translation | | | | | | | | |
| | | | elongation factor 2 | | | | | | | | |
| 181 | 22994 | CEP131 (SEQ ID | centrosomal protein | | | | | | 1 | | |
| | | NO: 181) | 131 kDa | | | | | | | | |
| 182 | 80342 | TRAF3IP3 (SEQ ID | TRAF3 interacting | | | | 1 | | | | |
| | | NO: 182) | protein 3, - | | | | | | | | |
| 183 | 3728 | JUP (SEQ ID | junction | | | | | | 1 | | |
| | | NO: 183) | plakoglobin | | | | | | | | |
| 184 | 6242 | RTKN (SEQ ID | rhotekin, junction | | | | | | 1 | | |
| | | NO: 184) | plakoglobin | | | | | | | | |
| 185 | 90993 | CREB3L1 (SEQ ID | cAMP responsive | | | | | | | | 1 |
| | | NO: 185) | element binding | | | | | | | | |
| | | | protein 3-like 1 | | | | | | | | |
| 186 | 5657 | PRTN3 (SEQ ID | Proteinase (PR3; | | | | | | 1 | | |
| | | NO: 186) | non recombinant) | | | | | | | | |
| 187 | 7481 | WNT11 (SEQ ID | wingless-type MMTV | | | | | 1 | | | |
| | | NO: 187) | integration site | | | | | | | | |
| | | | family, member 11 | | | | | | | | |
| 188 | 922 | CD5L (SEQ ID | CD5 molecule-like | | | | | | | | 1 |
| | | NO: 188) | | | | | | | | | |
| 189 | 1001 | CDH3 (SEQ ID | cadherin 3, type 1, | | | | | | | | |
| | | NO: 189) | P-cadherin | | | | | | | | |
| | | | (placental) | | | | | | | | |
| 190 | 8190 | MIA (SEQ ID | melanoma inhibitory | | | | | | | | 1 |
| | | NO: 190) | activity | | | | | | | | |
| 191 | 4102 | MAGEA3 (SEQ ID | MAGE family member | | | | | 1 | | | |
| | | NO: 191) | A3 | | | | | | | | |
| 192 | 54472 | TOLLIP (SEQ ID | toll interacting | | 1 | | | | | | |
| | | NO: 192) | protein | | | | | | | | |
| 193 | 5175 | PECAM1 (SEQ ID | platelet/endothelial | | | | | | | | |
| | | NO: 193) | cell adhesion | | | | | | | | |
| | | | molecule 1 | | | | | | | | |
| 194 | 57402 | S100A14 (SEQ ID | S100 calcium | | | | | | | | 1 |
| | | NO: 194) | binding protein A14 | | | | | | | | |
| 195 | 9826 | ARHGEF11 (SEQ ID | Rho guanine | 1 | | | | | | | |
| | | NO: 195) | nucleotide exchange | | | | | | | | |
| | | | factor (GEF) 11 | | | | | | | | |
| 196 | 5455 | POU3F3 (SEQ ID | POU class 3 | | | | | | | 1 | |
| | | NO: 196) | homeobox 3 | | | | | | | | |
| 197 | 112950 | MED8 (SEQ ID | mediator complex | | | | | | | | 1 |
| | | NO: 197) | subunit 8 | | | | | | | | |
| 198 | 1191 | CLU (SEQ ID | clusterin | | | | | | | | 1 |
| | | NO: 198) | | | | | | | | | |
| 199 | 7276 | TTR (SEQ ID | transthyretin | | | | | | | | 1 |
| | | NO: 199) | | | | | | | | | |
| 200 | 4858 | NOVA2 (SEQ ID | neuro-oncological | | | | 1 | | | | |
| | | NO: 200) | ventral antigen 2 | | | | | | | | |
| 201 | 5868 | RAB5A (SEQ ID | RAB5A, member RAS | | | | | | | 1 | |
| | | NO: 201) | oncogene family | | | | | | | | |
| 202 | 1511 | CTSG (SEQ ID | cathepsin G, small | | | | | | | 1 | |
| | | NO: 202) | nuclear | | | | | | | | |
| | | | ribonucleoprotein | | | | | | | | |
| | | | D3 polypeptide | | | | | | | | |
| | | | 18 kDa | | | | | | | | |
| 203 | 6634 | SNRPD3 (SEQ ID | small nuclear | | | | | | | 1 | |
| | | NO: 203) | ribonucleoprotein | | | | | | | | |
| | | | D3 polypeptide | | | | | | | | |
| | | | 18 kDa | | | | | | | | |
| 204 | 11140 | CDC37 (SEQ ID | cell division cycle | | | | | | | 1 | |
| | | NO: 204) | 37 | | | | | | | | |
| 205 | 3586 | IL10 (SEQ ID | interleukin 10 | | | | | 1 | | | |
| | | NO: 205) | | | | | | | | | |
| 206 | 84419 | C15orf48 (SEQ ID | chromosome 15 open | | | | | | | 1 | |
| | | NO: 206) | reading frame 48 | | | | | | | | |
| 207 | 3125 | HLA-DRB3 (SEQ ID | major | | 1 | | | | | | |
| | | NO: 207) | histocompatibility | | | | | | | | |
| | | | complex, class II, | | | | | | | | |
| | | | DR beta 3 | | | | | | | | |
| 208 | 1485 | CTAG1B (SEQ ID | cancer/testis | | | | | | | 1 | |
| | | NO: 208) | antigen 1B | | | | | | | | |
| 209 | 1485 | CTAG1B (SEQ ID | cancer/testis | | | | | | | 1 | |
| | | NO: 209) | antigen 1B | | | | | | | | |
| 210 | 4282 | MIF (SEQ ID | macrophage | 1 | | | | | | | |
| | | NO: 210) | migration | | | | | | | | |
| | | | inhibitory factor | | | | | | | | |
| | | | (glycosylation- | | | | | | | | |
| | | | inhibiting factor) | | | | | | | | |
| 211 | 1297 | COL9A1 (SEQ ID | collagen, type IX, | | 1 | | | | | | |
| | | NO: 211) | alpha 1 | | | | | | | | |
| 212 | 5777 | PTPN6 (SEQ ID | protein tyrosine | 1 | | | | | | | |
| | | NO: 212) | phosphatase, non- | | | | | | | | |
| | | | receptor type 6 | | | | | | | | |
| 213 | 4793 | NFKBIB (SEQ ID | nuclear factor of | | | | | | | | 1 |
| | | NO: 213) | kappa light | | | | | | | | |
| | | | polypeptide gene | | | | | | | | |
| | | | enhancer in B-cells | | | | | | | | |
| | | | inhibitor, beta | | | | | | | | |
| 214 | 4137 | MAPT (SEQ ID | microtubule- | | | | | | 1 | | |
| | | NO: 214) | associated protein | | | | | | | | |
| | | | tau | | | | | | | | |
| 215 | 1509 | CTSD (SEQ ID | cathepsin D | | | | | | 1 | | |
| | | NO: 215) | | | | | | | | | |
| 216 | 1485 | CTAG1B (SEQ ID | cancer/testis | | | | | | | | |
| | | NO: 216) | antigen 1B | | | | | | | | |
| 217 | 1485 | CTAG1B (SEQ ID | cancer/testis | | | | | | | | |
| | | NO: 217) | antigen 1B | | | | | | | | |
| 218 | 201161 | CENPV (SEQ ID | centromere protein | | | | | | 1 | | |
| | | NO: 218) | V | | | | | | | | |
| 219 | 6117 | RPA1 (SEQ ID | replication protein | 1 | | | | | | | |
| | | NO: 219) | A1, 70 kDa | | | | | | | | |
| 220 | 4103 | MAGEA4 (SEQ ID | MAGE family member | | | | | | | | |
| | | NO: 220) | A4 | | | | | | | | |
| 221 | 10563 | CXCL13 (SEQ ID | chemokine (C-X-C | | 1 | | | | | | |
| | | NO: 221) | motif) ligand 13 | | | | | | | | |
| 222 | 6351 | CCL4 (SEQ ID | chemokine (C-C | | | | 1 | | | | |
| | | NO: 222) | motif) ligand 4 | | | | | | | | |
| 223 | 7417 | VDAC2 (SEQ ID | voltage-dependent | | | | 1 | | | | |
| | | NO: 223) | anion channel 2 | | | | | | | | |
| 224 | 10643 | IGF2BP3 (SEQ ID | insulin-like growth | 1 | | | | | | | |
| | | NO: 224) | factor 2 mRNA | | | | | | | | |
| | | | binding protein 3 | | | | | | | | |
| 225 | 994 | CDC25B (SEQ ID | cell division cycle | 1 | | | | | | | |
| | | NO: 225) | 25B | | | | | | | | |
| 226 | 9240 | PNMA1 (SEQ ID | paraneoplastic Ma | | | | | | | | 1 |
| | | NO: 226) | antigen 1 | | | | | | | | |
| 227 | 156 | GRK2 (SEQ ID | G protein-coupled | | | | 1 | | | | |
| | | NO: 227) | receptor kinase 2 | | | | | | | | |
| 228 | 10071 | MUC12 (SEQ ID | mucin 12, cell | | | | 1 | | | | |
| | | NO: 228) | surface associated | | | | | | | | |
| 229 | 3326 | HSP90AB1 (SEQ ID | heat shock protein | | | | 1 | | | | |
| | | NO: 229) | 90 kDa alpha | | | | | | | | |
| | | | (cytosolic), class | | | | | | | | |
| | | | B member 1 | | | | | | | | |
| 230 | 64806 | IL25 (SEQ ID | interleukin 25 | | | | | | | | 1 |
| | | NO: 230) | | | | | | | | | |
| 231 | 286514 | MAGEB18 (SEQ ID | melanoma antigen | | | | | | | | 1 |
| | | NO: 231) | family B, 18 | | | | | | | | |
| 232 | 4150 | MAZ (SEQ ID | MYC-associated zinc | | | | 1 | | | | |
| | | NO: 232) | finger protein | | | | | | | | |
| | | | (purine-binding | | | | | | | | |
| | | | transcription | | | | | | | | |
| | | | factor) | | | | | | | | |
| 233 | 5899 | RALB (SEQ ID | v-ral simian | | | | | | | | |
| | | NO: 233) | leukemia viral | | | | | | | | |
| | | | oncogene homolog B | | | | | | | | |
| | | | (ras related; GTP | | | | | | | | |
| | | | binding protein) | | | | | | | | |
| 234 | 7918 | GPANK1 (SEQ ID | G patch domain and | | | | | | | 1 | |
| | | NO: 234) | ankyrin repeats 1 | | | | | | | | |
| 235 | 80310 | PDGFD (SEQ ID | platelet derived | | | | | | 1 | | |
| | | NO: 235) | growth factor D | | | | | | | | |
| 236 | 2670 | GFAP (SEQ ID | glial fibrillary | | 1 | | | | | | |
| | | NO: 236) | acidic protein | | | | | | | | |
| 237 | 55801 | IL26 (SEQ ID | interleukin 26 | | | | | | | | 1 |
| | | NO: 237) | | | | | | | | | |
| 238 | 7001 | PRDX2 (SEQ ID | peroxiredoxin 2 | | | | | | | 1 | |
| | | NO: 238) | | | | | | | | | |
| 239 | 128866 | CHMP4B (SEQ ID | charged | | | | 1 | | | | |
| | | NO: 239) | multivesicular body | | | | | | | | |
| | | | protein 4B | | | | | | | | |
| 240 | 7204 | TRIO (SEQ ID | trio Rho guanine | | 1 | | | | | | |
| | | NO: 240) | nucleotide exchange | | | | | | | | |
| | | | factor | | | | | | | | |
| 241 | 7134 | TNNC1 (SEQ ID | troponin C type 1 | | 1 | | | | | | |
| | | NO: 241) | (slow) | | | | | | | | |
| 242 | 652 | BMP4 (SEQ ID | bone morphogenetic | 1 | | | | | | | |
| | | NO: 242) | protein 4 | | | | | | | | |
| 243 | 203286 | ANKS6 (SEQ ID | ankyrin repeat and | | | | | | 1 | | |
| | | NO: 243) | sterile alpha motif | | | | | | | | |
| | | | domain containing 6 | | | | | | | | |
| 244 | 5529 | PPP2R5E (SEQ ID | protein phosphatase | | | | 1 | | | | |
| | | NO: 244) | 2, regulatory | | | | | | | | |
| | | | subunit B′, epsilon | | | | | | | | |
| | | | isoform | | | | | | | | |
| 245 | 1398 | CRK (SEQ ID | v-crk avian sarcoma | 1 | | | | | | | |
| | | NO: 245) | virus CT10 oncogene | | | | | | | | |
| | | | homolog | | | | | | | | |
| 246 | 332 | BIRC5 (SEQ ID | baculoviral IAP | | | | 1 | | | | |
| | | NO: 246) | repeat containing 5 | | | | | | | | |
| 247 | 23400 | ATP13A2 (SEQ ID | ATPase type 13A2 | 1 | | | | | | | |
| | | NO: 247) | | | | | | | | | |
| 248 | 2617 | GARS (SEQ ID | glycyl-tRNA | | | | 1 | | | | |
| | | NO: 248) | synthetase | | | | | | | | |
| 249 | 60 | ACTB (SEQ ID | actin, beta | | | | | | | | 1 |
| | | NO: 249) | | | | | | | | | |
| 250 | 4302 | MLLT6 (SEQ ID | myeloid/lymphoid or | | | | | | | | 1 |
| | | NO: 250) | mixed-lineage | | | | | | | | |
| | | | leukemia (trithorax | | | | | | | | |
| | | | homolog, | | | | | | | | |
| | | | Drosophila); | | | | | | | | |
| | | | translocated to, 6, | | | | | | | | |
| | | | actin, beta | | | | | | | | |
| 251 | 79714 | CCDC51 (SEQ ID | coiled-coil domain | | | | 1 | | | | |
| | | NO: 251) | containing 51 | | | | | | | | |
| 252 | 5535 | PPP3R2 (SEQ ID | protein phosphatase | | | | 1 | | | | |
| | | NO: 252) | 3 (formerly 2B) | | | | | | | | |
| 253 | 5155 | PDGFB (SEQ ID | platelet-derived | | | | | | 1 | | |
| | | NO: 253) | growth factor beta | | | | | | | | |
| | | | polypeptide | | | | | | | | |
| 254 | 55703 | POLR3B (SEQ ID | polymerase (RNA) | | | | | | | | 1 |
| | | NO: 254) | III (DNA directed) | | | | | | | | |
| | | | polypeptide B | | | | | | | | |
| 255 | 3853 | KRT6A (SEQ ID | keratin 6A, type II | | | | | | | | 1 |
| | | NO: 255) | | | | | | | | | |
| 256 | 2885 | GRB2 (SEQ ID | growth factor | | | | | | | | 1 |
| | | NO: 256) | receptor-bound | | | | | | | | |
| | | | protein 2 | | | | | | | | |
| 257 | 1284 | COL4A2 (SEQ ID | collagen, type IV, | | | | | | | | 1 |
| | | NO: 257) | alpha 2 | | | | | | | | |
| 258 | 2572 | GAD2 (SEQ ID | glutamate | | 1 | | | | | | |
| | | NO: 258) | decarboxylase 2 | | | | | | | | |
| | | | (pancreatic islets | | | | | | | | |
| | | | and brain, 65 kDa) | | | | | | | | |
| 259 | 80705 | TSGA10 (SEQ ID | testis specific, 10 | | | | 1 | | | | |
| | | NO: 259) | | | | | | | | | |
| 260 | 9807 | IP6K1 (SEQ ID | inositol | | 1 | | | | | | |
| | | NO: 260) | hexakisphosphate | | | | | | | | |
| | | | kinase 1 | | | | | | | | |
| 261 | 1981 | EIF4G1 (SEQ ID | eukaryotic | | | | | 1 | | | |
| | | NO: 261) | translation | | | | | | | | |
| | | | initiation factor 4 | | | | | | | | |
| | | | gamma, 1 | | | | | | | | |
| 262 | 10290 | SPEG (SEQ ID | SPEG complex locus | | | | | | | | |
| | | NO: 262) | | | | | | | | | |
| 263 | 1991 | ELANE (SEQ ID | elastase, | | | | 1 | | | | |
| | | NO: 263) | neutrophil | | | | | | | | |
| | | | expressed | | | | | | | | |
| 264 | 5055 | SERPINB2 (SEQ ID | serpin peptidase | | | | | | 1 | | |
| | | NO: 264) | inhibitor, clade B | | | | | | | | |
| | | | (ovalbumin), member 2 | | | | | | | | |
| 265 | 3446 | IFNA10 (SEQ ID | interferon, alpha | | | | | | | | 1 |
| | | NO: 265) | 10 | | | | | | | | |
| 266 | 3441 | IFNA4 (SEQ ID | interferon, alpha 4 | | | | | | | | 1 |
| | | NO: 266) | | | | | | | | | |
| 267 | 80152 | CENPT (SEQ ID | centromere protein | | | | | | | 1 | |
| | | NO: 267) | T | | | | | | | | |
| 268 | 1107 | CHD3 (SEQ ID | chromodomain | | 1 | | | | | | |
| | | NO: 268) | helicase DNA | | | | | | | | |
| | | | binding protein 3 | | | | | | | | |
| 269 | 25824 | PRDX5 (SEQ ID | peroxiredoxin 5 | | | | 1 | | | | |
| | | NO: 269) | | | | | | | | | |
| 270 | 11214 | AKAP13 (SEQ ID | A kinase (PRKA) | | 1 | | | | | | |
| | | NO: 270) | anchor protein 13 | | | | | | | | |
| 271 | 6626 | SNRPA (SEQ ID | U1-snRNP A | | | | 1 | | | | |
| | | NO: 271) | | | | | | | | | |
| 272 | 1993 | ELAVL2 (SEQ ID | ELAV like neuron- | | 1 | | | | | | |
| | | NO: 272) | specific RNA | | | | | | | | |
| | | | binding protein 2 | | | | | | | | |
| 273 | 1995 | ELAVL3 (SEQ ID | ELAV like neuron- | | 1 | | | | | | |
| | | NO: 273) | specific RNA | | | | | | | | |
| | | | binding protein 3, | | | | | | | | |
| | | | ELAV like neuron- | | | | | | | | |
| | | | specific RNA | | | | | | | | |
| | | | binding protein 2 | | | | | | | | |
| 274 | 720 | C4A (SEQ ID | complement | | | | | | | | 1 |
| | | NO: 274) | component 4A | | | | | | | | |
| | | | (Rodgers blood | | | | | | | | |
| | | | group) | | | | | | | | |
| 275 | 56475 | RPRM (SEQ ID | reprimo, TP53 | | | | | | | | 1 |
| | | NO: 275) | dependent G2 arrest | | | | | | | | |
| | | | mediator candidate | | | | | | | | |
| 276 | 118430 | MUCL1 (SEQ ID | mucin-like 1 | | | | 1 | | | | |
| | | NO: 276) | | | | | | | | | |
| 277 | 84957 | RELT (SEQ ID | RELT tumor necrosis | | 1 | | | | | | |
| | | NO: 277) | factor receptor | | | | | | | | |
| 278 | 5025 | P2RX4 (SEQ ID | purinergic receptor | | | | | | | | 1 |
| | | NO: 278) | P2X, ligand gated | | | | | | | | |
| | | | ion channel, 4 | | | | | | | | |
| 279 | 3646 | EIF3E (SEQ ID | eukaryotic | | | | | | | 1 | |
| | | NO: 279) | translation | | | | | | | | |
| | | | initiation factor | | | | | | | | |
| | | | 3, subunit E | | | | | | | | |
| 280 | 4582 | MUC1 (SEQ ID | mucin 1, cell | | | | 1 | | | | |
| | | NO: 280) | surface associated | | | | | | | | |
| 281 | 84365 | NIFK (SEQ ID | nucleolar protein | | | | | | | 1 | |
| | | NO: 281) | interacting with | | | | | | | | |
| | | | the FHA domain of | | | | | | | | |
| | | | MKI67 | | | | | | | | |
| 282 | 3004 | GZMM (SEQ ID | granzyme M | | | | | | 1 | | |
| | | NO: 282) | (lymphocyte met-ase | | | | | | | | |
| | | | 1) | | | | | | | | |
| 283 | 2160 | F11 (SEQ ID | coagulation factor | | | | | | | | 1 |
| | | NO: 283) | XI | | | | | | | | |
| 284 | 174 | AFP (SEQ ID | alpha-fetoprotein | | | | | | | | 1 |
| | | NO: 284) | | | | | | | | | |
| 285 | 3662 | IRF4 (SEQ ID | interferon | | | | 1 | | | | |
| | | NO: 285) | regulatory factor 4 | | | | | | | | |
| 286 | 5337 | PLD1 (SEQ ID | phospholipase D1, | | | | | | | | 1 |
| | | NO: 286) | phosphatidylcholine- | | | | | | | | |
| | | | specific | | | | | | | | |
| 287 | 25930 | PTPN23(formerly: | protein tyrosine | | | | | | | 1 | |
| | | SRPR) (SEQ ID | phosphatase, non- | | | | | | | | |
| | | NO: 287) | receptor type 23 | | | | | | | | |
| 288 | 7419 | VDAC3 (SEQ ID | voltage-dependent | | | | | | | | 1 |
| | | NO: 288) | anion channel 3 | | | | | | | | |
| 289 | 5921 | RASA1 (SEQ ID | RAS p21 protein | | | | | | | | 1 |
| | | NO: 289) | activator (GTPase | | | | | | | | |
| | | | activating protein) 1 | | | | | | | | |
| 290 | 8027 | STAM (SEQ ID | signal transducing | | | | | | | | 1 |
| | | NO: 290) | adaptor molecule | | | | | | | | |
| | | | (SH3 domain and | | | | | | | | |
| | | | ITAM motif) 1 | | | | | | | | |
| 291 | 2064 | ERBB2 (SEQ ID | erb-b2 receptor | | | | 1 | | | | |
| | | NO: 291) | tyrosine kinase 2 | | | | | | | | |
| 292 | 29108 | PYCARD (SEQ ID | PYD and CARD domain | 1 | | | | | | | |
| | | NO: 292) | containing | | | | | | | | |
| 293 | 4241 | MELTF (formerly: | melanotransferrin | | | | | | 1 | | |
| | | MFI2) (SEQ ID | | | | | | | | | |
| | | NO: 293) | | | | | | | | | |
| 294 | 2243 | FGA (SEQ ID | fibrinogen alpha | | | | 1 | | | | |
| | | NO: 294) | chain | | | | | | | | |
| 295 | 257106 | ARHGAP30 (SEQ ID | Rho GTPase | | | | 1 | | | | |
| | | NO: 295) | activating protein | | | | | | | | |
| | | | 30 | | | | | | | | |
| 296 | 2185 | PTK2B (SEQ ID | protein tyrosine | 1 | | | | | | | |
| | | NO: 296) | kinase 2 beta | | | | | | | | |
| 297 | 4599 | MX1 (SEQ ID | MX dynamin-like | | | | | | | 1 | |
| | | NO: 297) | GTPase 1 | | | | | | | | |
| 298 | 9447 | AIM2 (SEQ ID | absent in melanoma | | 1 | | | | | | |
| | | NO: 298) | 2 | | | | | | | | |
| 299 | 165 | AEBP1 (SEQ ID | AE binding protein | | 1 | | | | | | |
| | | NO: 299) | 1 | | | | | | | | |
| 300 | 7917 | BAG6 (SEQ ID | BCL2-associated | | | | | | | | |
| | | NO: 300) | athanogene 6 | | | | | | | | |
| 301 | 4846 | NOS3 (SEQ ID | nitric oxide | | | | 1 | | | | |
| | | NO: 301) | synthase 3 | | | | | | | | |
| | | | (endothelial cell) | | | | | | | | |
| 302 | 655 | BMP7 (SEQ ID | bone morphogenetic | | | | | | | 1 | |
| | | NO: 302) | protein 7, chordin | | | | | | | | |
| 303 | 8646 | CHRD (SEQ ID | chordin | | | | | | | 1 | |
| | | NO: 303) | | | | | | | | | |
| 304 | 27101 | CACYBP (SEQ ID | calcyclin binding | | | | 1 | | | | |
| | | NO: 304) | protein | | | | | | | | |
| 305 | 58498 | MYL7 (SEQ ID | myosin, light chain | | | | | | | 1 | |
| | | NO: 305) | 7, regulatory | | | | | | | | |
| 306 | 4286 | MITF (SEQ ID | microphthalmia- | | | | | | | | 1 |
| | | NO: 306) | associated | | | | | | | | |
| | | | transcription | | | | | | | | |
| | | | factor | | | | | | | | |
| 307 | 10537 | UBD (SEQ ID | ubiquitin D | | | | | | | | 1 |
| | | NO: 307) | | | | | | | | | |
| 308 | 312 | ANXA13 (SEQ ID | annexin A13 | | | | 1 | | | | |
| | | NO: 308) | | | | | | | | | |
| 309 | 3320 | HSP90AA1 (SEQ ID | heat shock protein | | | | | | | | 1 |
| | | NO: 309) | 90 kDa alpha | | | | | | | | |
| | | | (cytosolic), class | | | | | | | | |
| | | | A member 1 | | | | | | | | |
| 310 | 3960 | LGALS4 (SEQ ID | lectin, | | | | | | | 1 | |
| | | NO: 310) | galactoside- | | | | | | | | |
| | | | binding, soluble, 4 | | | | | | | | |
| 311 | 23135 | KDM6B (SEQ ID | lysine (K)-specific | | | | | | 1 | | |
| | | NO: 311) | demethylase 6B | | | | | | | | |
| 312 | 3181 | HNRNPA2B1 (SEQ ID | heterogeneous | | | | 1 | | | | |
| | | NO: 312) | nuclear | | | | | | | | |
| | | | ribonucleoprotein | | | | | | | | |
| | | | A2/B1 | | | | | | | | |
| 313 | 4869 | NPM1 (SEQ ID | nucleophosmin | 1 | | | | | | | |
| | | NO: 313) | (nucleolar | | | | | | | | |
| | | | phosphoprotein B23, | | | | | | | | |
| | | | numatrin) | | | | | | | | |
| 314 | 51135 | IRAK4 (SEQ ID | interleukin-1 | | | | | | | | 1 |
| | | NO: 314) | receptor-associated | | | | | | | | |
| | | | kinase 4 | | | | | | | | |
| 315 | 567 | B2M (SEQ ID | beta-2- | | | | | | | | 1 |
| | | NO: 315) | microglobulin | | | | | | | | |
| 316 | 1977 | EIF4E (SEQ ID | eukaryotic | | | | | | | 1 | |
| | | NO: 316) | translation | | | | | | | | |
| | | | initiation factor | | | | | | | | |
| | | | 4E | | | | | | | | |
| 317 | 6629 | SNRPB2 (SEQ ID | small nuclear | | | | | | 1 | | |
| | | NO: 317) | ribonucleoprotein | | | | | | | | |
| | | | polypeptide B2 | | | | | | | | |
| 318 | 708 | C1QBP (SEQ ID | complement | | | | 1 | | | | |
| | | NO: 318) | component 1, q | | | | | | | | |
| | | | subcomponent | | | | | | | | |
| | | | binding protein | | | | | | | | |
| 319 | 672 | BRCA1 (SEQ ID | breast cancer 1, | | | | 1 | | | | |
| | | NO: 319) | early onset | | | | | | | | |
| 320 | 3178 | HNRNPA1 (SEQ ID | heterogeneous | | | | 1 | | | | |
| | | NO: 320) | nuclear | | | | | | | | |
| | | | ribonucleoprotein | | | | | | | | |
| | | | A1 | | | | | | | | |
| 321 | 9506 | PAGE4 (SEQ ID | P antigen family, | | | | 1 | | | | |
| | | NO: 321) | member 4 (prostate | | | | | | | | |
| | | | associated) | | | | | | | | |
| 322 | 653220 | XAGE1B (SEQ ID | X antigen family, | | | | | | | | |
| | | NO: 322) | member 1B, X | | | | | | | | |
| | | | antigen family, | | | | | | | | |
| | | | member 1C, X | | | | | | | | |
| | | | antigen family, | | | | | | | | |
| | | | member 1E, X | | | | | | | | |
| | | | antigen family, | | | | | | | | |
| | | | member 1A | | | | | | | | |
| 323 | 653067 | XAGE1E (SEQ ID | X antigen family, | | | | | | | | |
| | | NO: 323) | member 1E | | | | | | | | |
| 324 | 3437 | IFIT3 (SEQ ID | interferon-induced | | | | | | | 1 | |
| | | NO: 324) | protein with | | | | | | | | |
| | | | tetratricopeptide | | | | | | | | |
| | | | repeats 3 | | | | | | | | |
| 325 | 29082 | CHMP4A (SEQ ID | charged | | | | | | | | 1 |
| | | NO: 325) | multivesicular body | | | | | | | | |
| | | | protein 4A | | | | | | | | |
| 326 | 10940 | POP1 (SEQ ID | processing of | | | | 1 | | | | |
| | | NO: 326) | precursor 1, | | | | | | | | |
| | | | ribonuclease P/MRP | | | | | | | | |
| | | | subunit | | | | | | | | |
| | | | (S. cerevisiae) | | | | | | | | |
| 327 | 6636 | SNRPF (SEQ ID | small nuclear | | | | | | | | 1 |
| | | NO: 327) | ribonucleoprotein | | | | | | | | |
| | | | polypeptide F | | | | | | | | |
| 328 | 3856 | KRT8 (SEQ ID | keratin 8, type II | | | | 1 | | | | |
| | | NO: 328) | | | | | | | | | |
| 329 | 801 | CALM1 (SEQ ID | calmodulin 1 | | | | | | | 1 | |
| | | NO: 329) | (phosphorylase | | | | | | | | |
| | | | kinase, delta) | | | | | | | | |
| 330 | 805 | CALM2 (SEQ ID | calmodulin 2 | | | | | | | 1 | |
| | | NO: 330) | (phosphorylase | | | | | | | | |
| | | | kinase, delta), | | | | | | | | |
| | | | calmodulin 1 | | | | | | | | |
| | | | (phosphorylase | | | | | | | | |
| | | | kinase, delta) | | | | | | | | |
| 331 | 6786 | STIM1 (SEQ ID | stromal interaction | | | | 1 | | | | |
| | | NO: 331) | molecule 1 | | | | | | | | |
| 332 | 81 | ACTN4 (SEQ ID | actinin, alpha 4 | 1 | | | | | | | |
| | | NO: 332) | | | | | | | | | |
| 333 | 6634 | SmD3 (SEQ ID | SmD3 | | | | | | | 1 | |
| | | NO: 333) | | | | | | | | | |
| 334 | 3710 | ITPR3 (SEQ ID | inositol 1,4,5- | | | | 1 | | | | |
| | | NO: 334) | trisphosphate | | | | | | | | |
| | | | receptor, type 3 | | | | | | | | |
| 335 | 115362 | GBP5 (SEQ ID | guanylate binding | | | | | | | | 1 |
| | | NO: 335) | protein 5 | | | | | | | | |
| 336 | 10573 | MRPL28 (SEQ ID | mitochondrial | | 1 | | | | | | |
| | | NO: 336) | ribosomal protein | | | | | | | | |
| | | | L28 | | | | | | | | |
| 337 | 10013 | HDAC6 (SEQ ID | histone deacetylase | | | | | | 1 | | |
| | | NO: 337) | 6 | | | | | | | | |
| 338 | 5105 | PCK1 (SEQ ID | phosphoenolpyruvate | | | | | | | | |
| | | NO: 338) | carboxykinase 1 | | | | | | | | |
| | | | (soluble) | | | | | | | | |
| 339 | 56300 | IL36G (SEQ ID | interleukin 36, | | 1 | | | | | | |
| | | NO: 339) | gamma | | | | | | | | |
| 340 | 598 | BCL2L1 (SEQ ID | BCL2-like 1 | | | | | | 1 | | |
| | | NO: 340) | | | | | | | | | |
| 341 | 3854 | KRT6B (SEQ ID | keratin 6B, type II | | | | | | | | 1 |
| | | NO: 341) | | | | | | | | | |
| 342 | 3880 | KRT19 (SEQ ID | keratin 19, type I | | | | | | | | |
| | | NO: 342) | | | | | | | | | |
| 343 | 6280 | S100A9 (SEQ ID | S100 calcium | | | | | | | | |
| | | NO: 343) | binding protein A9 | | | | | | | | |
| 344 | 3075 | CFH (SEQ ID | complement factor H | | | | | | | | 1 |
| | | NO: 344) | | | | | | | | | |
| 345 | 9470 | EIF4E2 (SEQ ID | eukaryotic | | | | | | | | |
| | | NO: 345) | translation | | | | | | | | |
| | | | initiation factor | | | | | | | | |
| | | | 4E family member 2 | | | | | | | | |
| 346 | 2810 | SFN (SEQ ID | stratifin | 1 | | | | | | | |
| | | NO: 346) | | | | | | | | | |
| 347 | 79650 | USB1 (SEQ ID | U6 snRNA biogenesis | | 1 | | | | | | |
| | | NO: 347) | 1 | | | | | | | | |
| 348 | 9500 | MAGED1 (SEQ ID | melanoma antigen | | | | | | | | 1 |
| | | NO: 348) | family D, 1 | | | | | | | | |
| 349 | 84968 | PNMA6A (SEQ ID | paraneoplastic Ma | | | | | | 1 | | |
| | | NO: 349) | antigen family | | | | | | | | |
| | | | member 6A | | | | | | | | |
| 350 | 6382 | SDC1 (SEQ ID | syndecan 1 | 1 | | | | | | | |
| | | NO: 350) | | | | | | | | | |
| 351 | 26525 | IL36RN (SEQ ID | interleukin 36 | 1 | | | | | | | |
| | | NO: 351) | receptor antagonist | | | | | | | | |
| 352 | 6631 | SNRPC (SEQ ID | U1-snRNP C | | | | | | | 1 | |
| | | NO: 352) | | | | | | | | | |
| 353 | 3902 | LAG3 (SEQ ID | lymphocyte- | | | | | | | | 1 |
| | | NO: 353) | activation gene 3 | | | | | | | | |
| 354 | 23061 | TBC1D9B (SEQ ID | TBC1 domain family, | | | | | | 1 | | |
| | | NO: 354) | member 9B (with | | | | | | | | |
| | | | GRAM domain) | | | | | | | | |
| 355 | 6923 | ELOB (formerly: | elongin B | | | | | | | | 1 |
| | | TCEB2) (SEQ ID | | | | | | | | | |
| | | NO: 355) | | | | | | | | | |
| 356 | 10963 | STIP1 (SEQ ID | stress-induced | | | | 1 | | | | |
| | | NO: 356) | phosphoprotein 1 | | | | | | | | |
| 357 | 6175 | RPLP0 (SEQ ID | ribosomal protein, | 1 | | | | | | | |
| | | NO: 357) | large, P0 | | | | | | | | |
| 358 | 3606 | IL18 (SEQ ID | interleukin 18 | | | | | | | | 1 |
| | | NO: 358) | | | | | | | | | |
| 359 | 1629 | DBT (SEQ ID | dihydrolipoamide | | | | | | | | |
| | | NO: 359) | branched chain | | | | | | | | |
| | | | transacylase E2 | | | | | | | | |
| 360 | 7405 | UVRAG (SEQ ID | UV radiation | | | | | | | | 1 |
| | | NO: 360) | resistance | | | | | | | | |
| | | | associated | | | | | | | | |
| 361 | 5154 | PDGFA (SEQ ID | platelet-derived | | | | | | | | 1 |
| | | NO: 361) | growth factor alpha | | | | | | | | |
| | | | polypeptide | | | | | | | | |
| 362 | 79441 | HAUS3 (SEQ ID | HAUS augmin-like | | | | | | | | 1 |
| | | NO: 362) | complex, subunit 3 | | | | | | | | |
| 363 | 10492 | SYNCRIP (SEQ ID | synaptotagmin | | | | | | 1 | | |
| | | NO: 363) | binding, | | | | | | | | |
| | | | cytoplasmic RNA | | | | | | | | |
| | | | interacting protein | | | | | | | | |
| 364 | 8566 | PDXK (SEQ ID | pyridoxal | | | 1 | | | | | |
| | | NO: 364) | (pyridoxine, | | | | | | | | |
| | | | vitamin B6) kinase | | | | | | | | |
| 365 | 2260 | FGFR1 (SEQ ID | fibroblast growth | | | 1 | | | | | |
| | | NO: 365) | factor receptor 1 | | | | | | | | |
| 366 | 5834 | PYGB (SEQ ID | phosphorylase, | | | 1 | | | | | |
| | | NO: 366) | glycogen; brain | | | | | | | | |
| 367 | 7186 | TRAF2 (SEQ ID | TNF receptor- | | | 1 | | | | | |
| | | NO: 367) | associated factor 2 | | | | | | | | |
| 368 | 84444 | DOT1L (SEQ ID | DOT1-like histone | | | 1 | | | | | |
| | | NO: 368) | H3K79 | | | | | | | | |
| | | | methyltransferase | | | | | | | | |

The GeneID is found on NCBI website available at www.ncbi.nlm.nih.gov. More information about the gene can be found by accessing the NCBI website and entering the GeneID or Gene Symbol, for instance. The sequence listing provided with the application contains the sequences of the above-identified antigen sequences encoded by the gene identified by the corresponding “Gene ID”.

### EXAMPLE 7

### Identification of Tumor-Associated Antigens in Prostate Cancer Patients

A tumor-associated antigen (TAA) is defined as an antigenic substance produced in the tumor, vascular or tumor surrounding tissue, which triggers an immune response in the host. A higher autoantibody level against a TAA is useful to determine the immuno-competence of cancer patients before treating a patient with an immuno-oncology (IO) therapy. Furthermore, TAA expressed in tumor cells or surrounding tissue are potential targets for use in cancer therapy. A further use of TAA is to diagnose cancer patients.

Group 1 comprises the best 49 tumor-associated antigens identified in prostate cancer. Group 1 antigens were identified by comparing the autoantibody levels in prostate cancer patients and with those in healthy control patients. Markers were identified by using the statistical technique Significance of microarrays in the R-programming language (SAMR). The strength of differences between the two test groups is computed as SAMR score_d. A positive fold-change value is indicative of higher autoantibody reactivity in the cancer group compared to healthy control samples. Shown below in Table 2 are the data on 49 TAA which elicit an immune response in prostate cancer.

TABLE 2
TAA identified in prostate cancer
(PCa) compared to healthy controls.

| Neu Nr | Gene | Gene | SAMR_PCa vs | SAMR_PCA vs |
|---|---|---|---|---|
| Patent | ID | Symbol | HC_Score.d. | HC_Fold.Change |
| 4 | 1026 | CDKN1A | 6.92 | 4.94 |
| 2 | 1437 | CSF2 | 5.26 | 2.72 |
| 14 | 4171 | MCM2 | 4.52 | 2.70 |
| 15 | 6494 | SIPA1 | 4.52 | 2.70 |
| 23 | 1493 | CTLA4 | 4.48 | 1.54 |
| 3 | 7408 | VASP | 4.41 | 2.04 |
| 56 | 91807 | MYLK3 | 4.34 | 2.73 |
| 219 | 6117 | RPA1 | 4.30 | 1.89 |
| 7 | 11315 | PARK7 | 4.11 | 2.03 |
| 59 | 1211 | CLTA | 3.82 | 1.81 |
| 19 | 9240 | PNMA1 | 3.78 | 1.59 |
| 20 | 64326 | RFWD2 | 3.70 | 1.61 |
| 94 | 10724 | MGEA5 | 3.56 | 1.52 |
| 136 | 10818 | FRS2 | 3.41 | 1.89 |
| 346 | 2810 | SFN | 3.36 | 1.53 |
| 195 | 9826 | ARHGEF11 | 3.29 | 1.54 |
| 350 | 6382 | SDC1 | 3.28 | 1.31 |
| 5 | 1655 | DDX5 | 3.17 | 1.52 |
| 332 | 81 | ACTN4 | 3.11 | 1.68 |
| 45 | 6195 | RPS6KA1 | 3.05 | 1.55 |
| 76 | 130617 | ZFAND2B | 3.01 | 1.41 |
| 351 | 26525 | IL36RN | 2.94 | 1.75 |
| 296 | 2185 | PTK2B | 2.91 | 1.59 |
| 35 | 1857 | DVL3 | 2.88 | 1.52 |
| 60 | 3312 | HSPA8 | 2.85 | 1.58 |
| 247 | 23400 | ATP13A2 | 2.85 | 1.48 |
| 99 | 4796 | TONSL | 2.81 | 1.66 |
| 78 | 841 | CASP8 | 2.80 | 1.49 |
| 70 | 10411 | RAPGEF3 | 2.80 | 1.76 |
| 212 | 5777 | PTPN6 | 2.78 | 1.39 |
| 152 | 10454 | TAB1 | 2.76 | 1.41 |
| 12 | 1027 | CDKN1B | 2.76 | 1.36 |
| 51 | 8320 | EOMES | 2.75 | 2.09 |
| 225 | 994 | CDC25B | 2.74 | 1.61 |
| 90 | 2316 | FLNA | 2.72 | 1.38 |
| 292 | 29108 | PYCARD | 2.69 | 1.43 |
| 87 | 27179 | IL36A | 2.66 | 1.49 |
| 210 | 4282 | MIF | 2.64 | 1.48 |
| 224 | 10643 | IGF2BP3 | 2.61 | 1.43 |
| 357 | 6175 | RPLP0 | 2.61 | 1.29 |
| 86 | 9275 | BCL7B | 2.59 | 1.71 |
| 11 | 2931 | GSK3A | 2.58 | 1.48 |
| 10 | 3304 | HSPA1B | 2.56 | 1.52 |
| 242 | 652 | BMP4 | 2.56 | 1.39 |
| 62 | 891 | CCNB1 | 2.56 | 1.52 |
| 172 | 6434 | TRA2B | 2.54 | 1.17 |
| 58 | 4436 | MSH2 | 2.54 | 1.45 |
| 245 | 1398 | CRK | 2.53 | 1.57 |
| 313 | 4869 | NPM1 | 2.51 | 1.22 |

The GeneID is found on NCBI website available at www.ncbi.nlm.nih.gov. More information about the gene can be found by accessing the NCBI website and entering the GeneID or Gene Symbol, for instance.

### EXAMPLE 8

### Measurement of Autoantibodies Induced in Prostate Cancer Patients Following PROSTVAC

Long-term positive effects on the overall survival of prostate cancer patients treated with the PROSTVAC vaccine may involve the stimulation of the humoral immune response in cancer patients. This may involve the induction of B cells and antibodies, which target additional antigens that are not directly included in the vaccine. This generation of a broader immune response is called antigen-spreading and could be important to achieve a sustainable anti-tumor response in patients.

Thus any new antibody and antigen, which is not part of the PROSTVAC vaccine, is a potential biomarker to measure the vaccination response in prostate cancer patients. In order to investigate if the vaccination with PROSTVAC can induce a post-treatment antibody response, the change in antibody levels between T0 (pre-treatment samples), T1 (3 month) and T2 (6 month) samples was analyzed. In total, antibody responses towards 842 antigens were analyzed. The post-treatment increase in the antibody levels from baseline was analyzed by correlation analysis using Pearson's correlaton (Study Day 0,1,2).

Table 3 includes the Pearson's r-value of 39 antigens, which induce a post-treatment antibody response in prostate cancer patients treated with PROSTVAC.

TABLE 3
Pearson's correlation of antigens and autoantibodies
with higher intensity levels following PROSTVAV treatment

| | | | r_in_PROSTVAC |
|---|---|---|---|
| ID | GeneID | Gene.Symbol | Study.Day |
| 270 | 11214 | AKAP13 | 0.51 |
| 3 | 7408 | VASP | 0.37 |
| 55 | 491 | ATP2B2 | 0.35 |
| 277 | 84957 | RELT | 0.34 |
| 347 | 79650 | USB1 | 0.34 |
| 343 | 6280 | S100A9 | 0.33 |
| 298 | 9447 | AIM2 | 0.31 |
| 339 | 56300 | IL36G | 0.29 |
| 30 | 163 | AP2B1 | 0.29 |
| 45 | 6195 | RPS6KA1 | 0.28 |
| 26 | 23151 | GRAMD4 | 0.27 |
| 211 | 1297 | COL9A1 | 0.27 |
| 29 | 6758 | SSX5 | 0.26 |
| 34 | 1743 | DLST | 0.26 |
| 165 | 483 | ATP1B3 | 0.26 |
| 236 | 2670 | GFAP | 0.26 |
| 158 | 307 | ANXA4 | 0.24 |
| 268 | 1107 | CHD3 | 0.24 |
| 11 | 2931 | GSK3A | 0.23 |
| 127 | 27332 | ZNF638 | 0.23 |
| 133 | 5493 | PPL | 0.23 |
| 241 | 7134 | TNNC1 | 0.23 |
| 48 | 3956 | LGALS1 | 0.23 |
| 40 | 208 | AKT2 | 0.23 |
| 272 | 1993 | ELAVL2 | 0.23 |
| 273 | 1995 | ELAVL3 | 0.23 |
| 336 | 10573 | MRPL28 | 0.22 |
| 50 | 3305 | HSPA1L | 0.22 |
| 81 | 310 | ANXA7 | 0.21 |
| 258 | 2572 | GAD2 | 0.21 |
| 192 | 54472 | TOLLIP | 0.21 |
| 207 | 3125 | HLA-DRB3 | 0.21 |
| 146 | 6374 | CXCL5 | 0.20 |
| 240 | 7204 | TRIO | 0.20 |
| 221 | 10563 | CXCL13 | 0.20 |
| 299 | 165 | AEBP1 | 0.20 |
| 27 | 3123 | HLA-DRB1 | 0.20 |
| 4 | 1026 | CDKN1A | 0.19 |
| 260 | 9807 | IP6K1 | 0.18 |

The GeneID is found on NCBI website available at www.ncbi.nlm.nih.gov. More information about the gene can be found by accessing the NCBI website and entering the GeneID or Gene Symbol, for instance.

### EXAMPLE 9

### Measurement of Autoantibodies Correlating with Time-to-Progression in Prostate Cancer Patients Treated with PROSTVAC

One of the reasons to terminate a patient's cancer therapy or to change the therapy is disease progression. The time from the beginning of the intervention until a patient shows signs of disease progression is called Progression-free survival (PFS). In PROSTVAC clinical studies a patient's PSA levels were determined pre-treatment and post-treatment. A biochemical progression was defined as a decrease in PSA levels of greater than or equal to 30% from baseline (T0) (https://clinicaltrials.gov/ct2/show/NCT00060528).

Biomarkers correlating with progression-free survival were calculated using Pearson's correlation.

Table 4 shows 50 markers correlating positively or negatively with progression-free survival in PROSTVAC treated patients.

Biomarkers correlating with progression-free survival were calculated using Pearson's correlation. Biomarkers with a positive r-value show positive correlation with progression-free survival and show higher intensity values in patients with longer PFS. Markers showing a positive correlation can be used to identify patients who are more likely to respond to PROSTVAC therapy.

In contrast, biomarkers with a negative r-value show a negative correlation with PFS and higher levels were found in patients with lower PFS. Patients who have higher levels of these markers are less likely to respond to therapy.

TABLE 4
Pearson's correlation coefficient of markers correlating
with progression−free survival in PROSTVAC treated patients.

| | | | r_in_PROSTVAC |
|---|---|---|---|
| | Gene | Gene | Progression- |
| ID | ID | Symbol | free survival |
| 105 | 6672 | Sp100 | 0.63 |
| 106 | 3959 | LGALS3BP | 0.63 |
| 107 | 1845 | DUSP3 | 0.58 |
| 26 | 23151 | GRAMD4 | 0.56 |
| 109 | 29115 | SAP30BP | 0.53 |
| 100 | 6757 | SSX2 | 0.52 |
| 2 | 1437 | CSF2 | 0.52 |
| 101 | 2920 | CXCL2 | 0.52 |
| 112 | 2335 | FN1 | 0.51 |
| 113 | 3002 | GZMB | 0.48 |
| 102 | 23367 | LARP1 | 0.47 |
| 114 | 140462 | ASB9 | 0.46 |
| 27 | 3123 | HLA-DRB1 | 0.46 |
| 115 | 7157 | TP53 | 0.45 |
| 116 | 4001 | LMNB1 | 0.45 |
| 103 | 2921 | CXCL3 | 0.45 |
| 28 | 5552 | SRGN | 0.44 |
| 118 | 2288 | FKBP4 | 0.43 |
| 104 | 4110 | MAGEA11 | 0.41 |
| 119 | 5533 | PPP3CC | 0.41 |
| 120 | 2157 | F8 | 0.41 |
| 6 | 6638 | SNRPN | 0.40 |
| 7 | 11315 | PARK7 | 0.40 |
| 108 | 1048 | CEACAM5 | 0.40 |
| 121 | 57568 | SIPA1L2 | 0.40 |
| 110 | 2633 | GBP1 | 0.40 |
| 111 | 593 | BCKDHA | 0.39 |
| 38 | 311 | ANXA11 | 0.39 |
| 122 | 23256 | SCFD1 | 0.38 |
| 123 | 55827 | DCAF6 | 0.38 |
| 29 | 6758 | SSX5 | 0.38 |
| 39 | 3624 | INHBA | 0.38 |
| 30 | 163 | AP2B1 | 0.38 |
| 40 | 208 | AKT2 | 0.38 |
| 31 | 7520 | XRCC5 | 0.36 |
| 41 | 1785 | DNM2 | 0.35 |
| 117 | 468 | ATF4 | 0.35 |
| 32 | 3554 | IL1R1 | 0.35 |
| 130 | 7157 | TP53 | 0.34 |
| 138 | 83593 | RASSF5 | 0.35 |
| 364 | 8566 | PDXK | −0.36 |
| 365 | 2260 | FGFR1 | −0.36 |
| 366 | 5834 | PYGB | −0.37 |
| 367 | 7186 | TRAF2 | −0.37 |
| 25 | 4107 | MAGEA8 | −0.38 |
| 368 | 84444 | DOT1L | −0.40 |
| 139 | 9909 | DENND4B | −0.42 |
| 36 | 3280 | HES1 | −0.44 |
| 140 | 1390 | CREM | −0.51 |
| 37 | 5585 | PKN1 | −0.53 |

The GeneID is found on NCBI website available at www.ncbi.nlm.nih.gov. More information about the gene can be found by accessing the NCBI website and entering the GeneID or Gene Symbol, for instance.

### EXAMPLE 10

### Measurement of Autoantibodies Correlating with OVERALL SURVIVAL in Prostate Cancer Patients Treated with PROSTVAC

An important clinical outcome measure in clinical trials is the Overall Survival (OS). The overall survival is defined as the date of on-study to the date of death from any cause or last follow-up.

Biomarkers correlating with OS were calculated using Pearson's correlation. Biomarkers with a positive r-value show positive correlation with OS and show higher intensity values in patients with longer OS. These markers can be used to identify patients who have a better overall survival time and may be more likely to benefit from PROSTVAC therapy.

In contrast, biomarkers with a negative r-value show a negative correlation with OS and higher levels were found in patients with lower OS.

Table 5 shows 70 markers correlating positively or negatively with OS in PROSTVAC treated patients.

TABLE 5
Pearson's correlation coefficient of markers
correlating with OS in PROSTVAC treated patients.

| | Gene | Gene | r_in_Prostvac |
|---|---|---|---|
| ID | ID | Symbol | Overall Survival |
| 164 | 23032 | USP33 | 0.57 |
| 49 | 79155 | TNIP2 | 0.57 |
| 168 | 7343 | UBTF | 0.56 |
| 161 | 25865 | PRKD2 | 0.52 |
| 280 | 4582 | MUC1 | 0.52 |
| 223 | 7417 | VDAC2 | 0.46 |
| 182 | 80342 | TRAF3IP3 | 0.46 |
| 16 | 5137 | PDE1C | 0.45 |
| 177 | 23396 | PIP5K1C | 0.45 |
| 2 | 1437 | CSF2 | 0.45 |
| 229 | 3326 | HSP90AB1 | 0.45 |
| 135 | 2266 | FGG | 0.45 |
| 41 | 1785 | DNM2 | 0.44 |
| 24 | 9133 | CCNB2 | 0.42 |
| 248 | 2617 | GARS | 0.41 |
| 31 | 7520 | XRCC5 | 0.41 |
| 174 | 3146 | HMGB1 | 0.40 |
| 1 | 3620 | IDO1 | 0.40 |
| 39 | 3624 | INHBA | 0.39 |
| 85 | 8490 | RGS5 | 0.39 |
| 301 | 4846 | NOS3 | 0.39 |
| 263 | 1991 | ELANE | 0.36 |
| 154 | 8971 | H1FX | 0.35 |
| 52 | 9173 | IL1RL1 | 0.35 |
| 251 | 79714 | CCDC51 | 0.35 |
| 291 | 2064 | ERBB2 | 0.35 |
| 304 | 27101 | CACYBP | 0.35 |
| 150 | 29968 | PSAT1 | 0.22 |
| 63 | 30848 | CTAG2 | −0.20 |
| 64 | 30848 | CTAG2 | −0.20 |
| 356 | 10963 | STIP1 | −0.32 |
| 156 | 629 | CFB | −0.35 |
| 334 | 3710 | ITPR3 | −0.35 |
| 252 | 5535 | PPP3R2 | −0.35 |
| 276 | 118430 | MUCL1 | −0.35 |
| 5 | 1655 | DDX5 | −0.35 |
| 244 | 5529 | PPP2R5E | −0.35 |
| 70 | 10411 | RAPGEF3 | −0.36 |
| 78 | 841 | CASP8 | −0.36 |
| 294 | 2243 | FGA | −0.36 |
| 321 | 9506 | PAGE4 | −0.36 |
| 239 | 128866 | CHMP4B | −0.37 |
| 222 | 6351 | CCL4 | −0.37 |
| 145 | 3566 | IL4R | −0.37 |
| 37 | 5585 | PKN1 | −0.38 |
| 71 | 8517 | IKBKG | −0.38 |
| 28 | 5552 | SRGN | −0.38 |
| 21 | 154 | ADRB2 | −0.38 |
| 246 | 332 | BIRC5 | −0.39 |
| 312 | 3181 | HNRNPA2B1 | −0.39 |
| 86 | 9275 | BCL7B | −0.39 |
| 271 | 6626 | SNRPA | −0.40 |
| 227 | 156 | GRK2 | −0.40 |
| 259 | 80705 | TSGA10 | −0.40 |
| 318 | 708 | C1QBP | −0.40 |
| 326 | 10940 | POP1 | −0.41 |
| 331 | 6786 | STIM1 | −0.41 |
| 129 | 8648 | NCOA1 | −0.42 |
| 228 | 10071 | MUC12 | −0.43 |
| 269 | 25824 | PRDX5 | −0.43 |
| 295 | 257106 | ARHGAP30 | −0.44 |
| 79 | 843 | CASP10 | −0.44 |
| 320 | 3178 | HNRNPA1 | −0.45 |
| 87 | 27179 | IL36A | −0.46 |
| 319 | 672 | BRCA1 | −0.47 |
| 328 | 3856 | KRT8 | −0.47 |
| 308 | 312 | ANXA13 | −0.48 |
| 285 | 3662 | IRF4 | −0.51 |
| 200 | 4858 | NOVA2 | −0.52 |
| 232 | 4150 | MAZ | −0.55 |

The GeneID is found on NCBI website available at www.ncbi.nlm.nih.gov. More information about the gene can be found by accessing the NCBI website and entering the GeneID or Gene Symbol, for instance.

### EXAMPLE 11

### Identification and Measurement of Antibodies Targeting Tumor-Associated Antigens and Self-Antigens in Prostate Cancer Patients Treated with PROSTVAC Plus Ipilimumab

Although PROSTVAC vaccination has been shown to improve the overall survival of prostate cancer patients, some patients experienced progression or relapse of the disease. There is evidence that cytotoxic T cells upregulate the T-lymphocyte-associated protein 4 (CTLA4), a negative regulatory molecule. Ipilimumab (Bristol-Myers Squibb, New York, N.Y., USA) is an antagonistic anti-CTLA4 monoclonal antibody that blocks the activity of CTLA4. Ipilimumab has been assessed in the treatment of prostate cancer, in which a minority (about 20%) of patients had significant PSA declines. Clinical data suggest, that combining immune checkpoint inhibition with therapeutic cancer vaccines, has the potential to improve the proportion of patients seeing long-term durable responses with these therapies.

In a phase I clinical trial 30 study participants with metastatic castration-resistant prostate cancer (mCRPC) were treated with PROSTVAC and escalating doses of ipilimumab (Madan et al., 2012). Serum samples from 24 patients treated with PROSTVAC plus ipilimumab were tested for the presence of autoantibodies against 842 preselected antigens Samples were collected prior to treatment (T0 samples) and two timepoints during treatment. The T1 corresponds to 90 days (3 month) and the T2 samples corresponds to 180 days (6 month).

### EXAMPLE 12

### Measurement of Autoantibodies Induced in Prostate Cancer Patients Following PROSTVAC Plus Ipilimumab

Long-term positive effects on the overall survival of prostate cancer patients treated with the PROSTVAC plus Ipilimumab may involve the stimulation of the humoral immune response in cancer patients. This may involve the induction of B cells and antibodies, which target additional antigens that are not directly included in the vaccine. This generation of a broader immune response is called antigen-spreading and could be important to achieve a sustainable anti-tumor response in patients.

Thus, any new antibody and antigen, which is not part of the PROSTVAC plus Ipilimumab treatment regime, is a potential biomarker to measure the vaccination response in prostate cancer patients. In order to investigate if PROSTVAC plus Ipilimumab can induce a post-treatment antibody response, the change in antibody levels between T0 (pre-treatment samples) and T1 (3 month) and T2 (6 month) samples was analyzed. In total, antibody responses towards 842 antigens were analyzed. The post-treatment increase in the antibody levels from baseline was analyzed by correlation analysis using Pearson's correlaton (Study Day 0,1,2).

Furthermore, the post-treatment samples T1 and T2 were compared to T0 samples using SAMR.

Table 6 includes the Pearson's r-value of 25 antigens, which induce a post-treatment antibody response in prostate cancer patients treated with PROSTVAC plus Ipilimumab.

TABLE 6
Markers induced by PROSTVAC plus Ipilimumab treatment

| | Gene | Gene | r-value | SAMR_pre- | SAMR_pre- |
|---|---|---|---|---|---|
| ID | ID | Symbol | Study.Day | post_treatment_Score.d. | post_treatment_Fold.Change |
| 4 | 1026 | CDKN1A | 0.10 | 4.19 | 3.80 |
| 2 | 1437 | CSF2 | 0.54 | 2.82 | 2.30 |
| 3 | 7408 | VASP | 0.63 | 3.51 | 2.26 |
| 191 | 4102 | MAGEA3 | 0.18 | 1.68 | 1.58 |
| 48 | 3956 | LGALS1 | 0.25 | 2.14 | 1.52 |
| 62 | 891 | CCNB1 | 0.28 | 1.75 | 1.51 |
| 205 | 3586 | IL10 | 0.17 | 1.54 | 1.44 |
| 137 | 3306 | HSPA2 | 0.39 | 1.73 | 1.42 |
| 155 | 7494 | XBP1 | 0.43 | 1.41 | 1.40 |
| 175 | 7167 | TPI1 | 0.14 | 1.53 | 1.34 |
| 50 | 3305 | HSPA1L | 0.36 | 1.59 | 1.31 |
| 60 | 3312 | HSPA8 | 0.37 | 1.26 | 1.31 |
| 42 | 729447 | GAGE2A | 0.22 | 0.92 | 1.26 |
| 43 | 729408 | GAGE2D | 0.22 | 0.92 | 1.26 |
| 44 | 26749 | GAGE2E | 0.22 | 0.92 | 1.26 |
| 134 | 5957 | RCVRN | 0.28 | 1.11 | 1.23 |
| 23 | 1493 | CTLA4 | 0.33 | 1.31 | 1.20 |
| 95 | 10285 | SMNDC1 | 0.24 | 0.55 | 1.16 |
| 261 | 1981 | EIF4G1 | 0.31 | 1.05 | 1.15 |
| 173 | 30827 | CXXC1 | 0.28 | 0.73 | 1.14 |
| 132 | 1639 | DCTN1 | 0.25 | 0.37 | 1.08 |
| 9 | 10015 | PDCD6IP | 0.27 | 0.24 | 1.05 |
| 187 | 7481 | WNT11 | 0.32 | 0.19 | 1.04 |
| 71 | 8517 | IKBKG | 0.24 | 0.17 | 1.03 |
| 36 | 3280 | HES1 | 0.25 | −0.42 | 0.90 |

The GeneID is found on NCBI website available at www.ncbi.nlm.nih.gov. More information about the gene can be found by accessing the NCBI website and entering the GeneID or Gene Symbol, for instance.

### EXAMPLE 13

### Measurement of Autoantibodies Correlating with the Predicted Median OS-Halabi in Prostate Cancer Patients Treated with PROSTVAC Plus Ipilimumab

One of the reasons to terminate a patient's cancer therapy or to change the therapy is disease progression. The predicted median overall survival (OS) by the Halabi nomogram is prognostic model for patients with metastatic castration-resistant prostate cancer (mCRPC) that can be used to compute individual predicted survival probability at different time points (Halabi et al., 2014).

Biomarkers correlating with OS-Halabi were calculated using Pearson's correlation.

Table 7 shows 64 markers correlating positively or negatively with OS-Halabi in PROSTVAC plus Ipilimumab treated patients.

TABLE 7
Pearson's correlation coefficient of markers correlating
with OS-Halabi in PROSTVAC plus Ipilimumab treated patients.

| | Gene | Gene | |
|---|---|---|---|
| ID | ID | Symbol | r_in_prostvac_ipi_Best.Response |
| 89 | 1 | A1BG | 0.49 |
| 66 | 64763 | ZNF574 | 0.48 |
| 85 | 8490 | RGS5 | 0.47 |
| 73 | 283431 | GAS2L3 | 0.46 |
| 58 | 4436 | MSH2 | 0.46 |
| 215 | 1509 | CTSD | 0.45 |
| 54 | 23646 | PLD3 | 0.44 |
| 47 | 3397 | ID1 | 0.43 |
| 157 | 1287 | COL4A5 | 0.42 |
| 282 | 3004 | GZMM | 0.42 |
| 183 | 3728 | JUP | 0.40 |
| 184 | 6242 | RTKN | 0.40 |
| 142 | 5934 | RBL2 | 0.40 |
| 69 | 2045 | EPHA7 | 0.39 |
| 82 | 1060 | CENPC | 0.39 |
| 143 | 3611 | ILK | 0.38 |
| 72 | 5223 | PGAM1 | 0.38 |
| 293 | 4241 | MELTF | 0.38 |
| | | (formerly: | |
| | | MFI2) | |
| 19 | 9240 | PNMA1 | 0.37 |
| 33 | 6625 | SNRNP70 | 0.37 |
| 153 | 65264 | UBE2Z | 0.37 |
| 264 | 5055 | SERPINB2 | 0.36 |
| 1 | 3620 | IDO1 | 0.36 |
| 96 | 10000 | AKT3 | 0.36 |
| 22 | 5971 | RELB | 0.36 |
| 235 | 80310 | PDGFD | 0.35 |
| 354 | 23061 | TBC1D9B | 0.35 |
| 20 | 64326 | RFWD2 | 0.35 |
| 17 | 4843 | NOS2 | 0.35 |
| 253 | 5155 | PDGFB | 0.35 |
| 97 | 5801 | PTPRR | 0.35 |
| 337 | 10013 | HDAC6 | 0.35 |
| 63 | 30848 | CTAG2 | −0.23 |
| 64 | 30848 | CTAG2 | −0.23 |
| 179 | 1938 | EEF2 | −0.35 |
| 180 | 1938 | EEF2 | −0.35 |
| 65 | 1387 | CREBBP | −0.36 |
| 243 | 203286 | ANKS6 | −0.36 |
| 46 | 273 | AMPH | −0.36 |
| 49 | 79155 | TNIP2 | −0.36 |
| 317 | 6629 | SNRPB2 | −0.36 |
| 218 | 201161 | CENPV | −0.36 |
| 311 | 23135 | KDM6B | −0.37 |
| 349 | 84968 | PNMA6A | −0.37 |
| 214 | 4137 | MAPT | −0.37 |
| 88 | 5906 | RAP1A | −0.38 |
| 68 | 59067 | IL21 | −0.38 |
| 181 | 22994 | CEP131 | −0.38 |
| 51 | 8320 | EOMES | −0.39 |
| 363 | 10492 | SYNCRIP | −0.40 |
| 16 | 5137 | PDE1C | −0.40 |
| 18 | 7299 | TYR | −0.42 |
| 340 | 598 | BCL2L1 | −0.42 |
| 11 | 2931 | GSK3A | −0.43 |
| 57 | 30850 | CDR2L | −0.43 |
| 84 | 283748 | PLA2G4D | −0.45 |
| 186 | 5657 | PRTN3 | −0.46 |
| 98 | 2099 | ESR1 | −0.46 |
| 8 | 8503 | PIK3R3 | −0.48 |
| 83 | 3161 | HMMR | −0.49 |
| 91 | 6464 | SHC1 | −0.49 |
| 25 | 4107 | MAGEA8 | −0.50 |

The GeneID is found on NCBI website available a www.ncbi.nlm.nih.gov. More information about the gene can be found by accessing the NCBI website and entering the GeneID or Gene Symbol, for instance.

### EXAMPLE 14

### Measurement of Autoantibodies Correlating with OVERALL SURVIVAL in Prostate Cancer Patients Treated with PROSTVAC

Biomarkers correlating with OS were calculated using Pearson's correlation. Biomarkers with a positive r-value show positive correlation with OS and show higher intensity values in patients with longer OS. These markers can be used to identify patients who have a better overall survival time and may be more likely to benefit from PROSTVAC plus Ipilimumab therapy.

In contrast, biomarkers with a negative r-value show a negative correlation with OS and higher levels were found in patients with lower OS.

Table 8 shows 70 markers correlating positively or negatively with OS in PROSTVAC treated patients.

TABLE 8
Markers correlating with OS in PROSTVAC
plus Ipilimumab treated patients.

| | Gene | Gene | |
|---|---|---|---|
| ID | ID | Symbol | r_in_prostvac_ipi_Overall.Survival |
| 33 | 6625 | SNRNP70 | 0.68 |
| 22 | 5971 | RELB | 0.59 |
| 47 | 3397 | ID1 | 0.55 |
| 96 | 10000 | AKT3 | 0.55 |
| 66 | 64763 | ZNF574 | 0.52 |
| 163 | 1174 | AP1S1 | 0.52 |
| 73 | 283431 | GAS2L3 | 0.52 |
| 333 | 6634 | SmD3 | 0.51 |
| 97 | 5801 | PTPRR | 0.50 |
| 238 | 7001 | PRDX2 | 0.49 |
| 13 | 26022 | TMEM98 | 0.47 |
| 82 | 1060 | CENPC | 0.45 |
| 17 | 4843 | NOS2 | 0.45 |
| 176 | 80184 | CEP290 | 0.45 |
| 69 | 2045 | EPHA7 | 0.44 |
| 74 | 5937 | RBMS1 | 0.42 |
| 14 | 4171 | MCM2 | 0.42 |
| 15 | 6494 | SIPA1 | 0.42 |
| 67 | 408 | ARRB1 | 0.41 |
| 9 | 10015 | PDCD6IP | 0.41 |
| 169 | 4000 | LMNA | 0.41 |
| 54 | 23646 | PLD3 | 0.41 |
| 267 | 80152 | CENPT | 0.40 |
| 206 | 84419 | C15orf48 | 0.40 |
| 89 | 1 | A1BG | 0.40 |
| 281 | 84365 | NIFK | 0.40 |
| 72 | 5223 | PGAM1 | 0.40 |
| 5 | 1655 | DDX5 | 0.39 |
| 80 | 3908 | LAMA2 | 0.39 |
| 329 | 801 | CALM1 | 0.38 |
| 330 | 805 | CALM2 | 0.38 |
| 196 | 5455 | POU3F3 | 0.38 |
| 305 | 58498 | MYL7 | 0.38 |
| 279 | 3646 | EIF3E | 0.38 |
| 171 | 2870 | GRK6 | 0.37 |
| 352 | 6631 | SNRPC | 0.37 |
| 1 | 3620 | IDO1 | 0.37 |
| 55 | 491 | ATP2B2 | 0.36 |
| 316 | 1977 | EIF4E | 0.36 |
| 201 | 5868 | RAB5A | 0.35 |
| 208 | 1485 | CTAG1B | 0.24 |
| 209 | 1485 | CTAG1B | 0.24 |
| 324 | 3437 | IFIT3 | 0.24 |
| 126 | 2919 | CXCL1 | 0.24 |
| 151 | 4088 | SMAD3 | 0.20 |
| 234 | 7918 | GPANK1 | −0.35 |
| 25 | 4107 | MAGEA8 | −0.35 |
| 16 | 5137 | PDE1C | −0.35 |
| 24 | 9133 | CCNB2 | −0.35 |
| 8 | 8503 | PIK3R3 | −0.35 |
| 18 | 7299 | TYR | −0.36 |
| 204 | 11140 | CDC37 | −0.37 |
| 202 | 1511 | CTSG | −0.37 |
| 203 | 6634 | SNRPD3 | −0.37 |
| 302 | 655 | BMP7 | −0.37 |
| 303 | 8646 | CHRD | −0.37 |
| 98 | 2099 | ESR1 | −0.37 |
| 297 | 4599 | Mχ1 | −0.38 |
| 6 | 6638 | SNRPN | −0.38 |
| 287 | 25930 | PTPN23 | −0.38 |
| | | (formerly: | |
| | | SRPR) | |
| 310 | 3960 | LGALS4 | −0.38 |
| 75 | 6181 | RPLP2 | −0.38 |
| 57 | 30850 | CDR2L | −0.38 |
| 167 | 4069 | LYZ | −0.39 |
| 53 | 6624 | FSCN1 | −0.41 |
| 91 | 6464 | SHC1 | −0.42 |
| 84 | 283748 | PLA2G4D | −0.43 |
| 162 | 10938 | EHD1 | −0.44 |
| 65 | 1387 | CREBBP | −0.49 |
| 83 | 3161 | HMMR | −0.55 |

The GeneID is found on NCBI website available at www.ncbi.nlm.nih.gov. More information about the gene can be found by accessing the NCBI website and entering the GeneID or Gene Symbol, for instance.

### EXAMPLE 15

### Identification of Biomarkers Associated with Immune-Related Adverse Effects (irAE) in PROSTVAC Plus Ipilimumab Treated Prostate Cancer Patients

Despite important clinical benefits, checkpoint inhibitors area associated with immune-related adverse events (irAEs) The mechanisms by which checkpoint inhibitors induce irAEs are not completely understood. It is believed that by blocking negative checkpoints a general immunologic enhancement occurs. It is also possible that by unleashing the immune-checkpoints that control tolerance, autoreactive lymphocytes are activated, which could be either T cells or B cells. It is well known that in autoimmune diseases autoreactive B cells produce autoantibodies that can induce tissue damage via ADCC. Thus, epitope spreading towards self-antigens may be an indicator for irAEs.

Autoantibodies correlating with irAEs were identified by Pearson's correlation analysis and SAMR.

Table 9 includes 87 biomarkers that are associated with irAE in PROSTVAC plus ipilimumab treated prostate cancer patients.

These biomarkers may be used to predict irAE in baseline samples of patients and prior to therapy or are induced following treatment.

TABLE 9
Biomarkers of irAE in patients treated
with PROSTVAC plus ipilimumab.

| | Gene | Gene | Pear- | SAMR | |
|---|---|---|---|---|---|
| ID | ID | Symbol | son'r | score.d. | SAMR_Fold.Change |
| 314 | 51135 | IRAK4 | 0.56 | 4.67 | 3.35 |
| 185 | 90993 | CREB3L1 | 0.53 | 3.91 | 1.95 |
| 249 | 60 | ACTB | 0.47 | 3.85 | 3.15 |
| 250 | 4302 | MLLT6 | 0.47 | 3.85 | 3.15 |
| 341 | 3854 | KRT6B | 0.46 | 3.32 | 1.87 |
| 52 | 9173 | IL1RL1 | 0.46 | 3.27 | 1.80 |
| 76 | 130617 | ZFAND2B | 0.46 | 3.15 | 1.66 |
| 14 | 4171 | MCM2 | 0.45 | 3.83 | 6.11 |
| 15 | 6494 | SIPA1 | 0.45 | 3.83 | 6.11 |
| 128 | 5034 | P4HB | 0.43 | 3.45 | 3.02 |
| 213 | 4793 | NFKBIB | 0.43 | 3.38 | 2.64 |
| 74 | 5937 | RBMS1 | 0.43 | 3.45 | 3.16 |
| 13 | 26022 | TMEM98 | 0.43 | 3.32 | 2.43 |
| 17 | 4843 | NOS2 | 0.42 | 3.21 | 2.22 |
| 226 | 9240 | PNMA1 | 0.42 | 3.11 | 2.03 |
| 178 | 3875 | KRT18 | 0.42 | 3.24 | 2.46 |
| 61 | 9094 | UNC119 | 0.41 | 3.28 | 2.98 |
| 1 | 3620 | IDO1 | 0.40 | 3.03 | 2.20 |
| 362 | 79441 | HAUS3 | 0.39 | 3.12 | 3.10 |
| 20 | 64326 | RFWD2 | 0.39 | 2.89 | 2.04 |
| 325 | 29082 | CHMP4A | 0.37 | 2.84 | 2.15 |
| 290 | 8027 | STAM | 0.37 | 2.68 | 1.75 |
| 148 | 26037 | SIPA1L1 | 0.37 | 2.83 | 2.28 |
| 12 | 1027 | CDKN1B | 0.37 | 2.67 | 1.82 |
| 257 | 1284 | COL4A2 | 0.37 | 2.33 | 1.40 |
| 32 | 3554 | IL1R1 | 0.37 | 2.66 | 1.79 |
| 255 | 3853 | KRT6A | 0.37 | 2.61 | 1.71 |
| 289 | 5921 | RASA1 | 0.37 | 2.88 | 2.71 |
| 81 | 310 | ANXA7 | 0.36 | 2.37 | 1.45 |
| 124 | 60560 | NAA35 | 0.36 | 2.75 | 2.11 |
| 9 | 10015 | PDCD6IP | 0.36 | 2.69 | 2.11 |
| 194 | 57402 | S100A14 | 0.35 | 2.56 | 1.78 |
| 95 | 10285 | SMNDC1 | 0.35 | 2.83 | 3.25 |
| 80 | 3908 | LAMA2 | 0.35 | 2.44 | 1.61 |
| 170 | 80184 | CEP290 | 0.35 | 2.69 | 2.35 |
| 93 | 4802 | NFYC | 0.35 | 2.64 | 2.16 |
| 254 | 55703 | POLR3B | 0.34 | 2.59 | 2.13 |
| 144 | 672 | BRCA1 | 0.34 | 2.60 | 2.20 |
| 131 | 1487 | CTBP1 | 0.34 | 2.60 | 2.20 |
| 92 | 5705 | PSMC5 | 0.34 | 2.67 | 2.81 |
| 10 | 3304 | HSPA1B | 0.33 | 2.54 | 2.12 |
| 160 | 10718 | NRG3 | 0.33 | 2.61 | 2.58 |
| 46 | 273 | AMPH | 0.33 | 2.55 | 2.22 |
| 355 | 6923 | ELOB | 0.33 | 2.52 | 2.20 |
| | | (formerly: | | | |
| | | TCEB2) | | | |
| 306 | 4286 | MITF | 0.33 | 2.52 | 2.20 |
| 141 | 2934 | GSN | 0.33 | 2.50 | 2.29 |
| 231 | 286514 | MAGEB18 | 0.32 | 2.43 | 2.10 |
| 315 | 567 | B2M | −0.35 | −2.24 | 0.70 |
| 344 | 3075 | CFH | −0.35 | −2.31 | 0.68 |
| 188 | 922 | CD5L | −0.35 | −1.70 | 0.85 |
| 283 | 2160 | F11 | −0.35 | −1.93 | 0.81 |
| 237 | 55801 | IL26 | −0.36 | −1.90 | 0.82 |
| 275 | 56475 | RPRM | −0.36 | −2.44 | 0.65 |
| 256 | 2885 | GRB2 | −0.37 | −2.50 | 0.64 |
| 75 | 6181 | RPLP2 | −0.37 | −2.52 | 0.64 |
| 361 | 5154 | PDGFA | −0.37 | −2.62 | 0.59 |
| 159 | 23299 | BICD2 | −0.37 | −2.71 | 0.55 |
| 286 | 5337 | PLD1 | −0.38 | −2.69 | 0.60 |
| 197 | 112950 | MED8 | −0.39 | −3.06 | 0.37 |
| 53 | 6624 | FSCN1 | −0.39 | −2.30 | 0.75 |
| 190 | 8190 | MIA | −0.39 | −2.75 | 0.59 |
| 6 | 6638 | SNRPN | −0.40 | −2.06 | 0.81 |
| 278 | 5025 | P2RX4 | −0.40 | −1.81 | 0.86 |
| 307 | 10537 | UBD | −0.40 | −2.88 | 0.54 |
| 59 | 1211 | CLTA | −0.41 | −3.26 | 0.33 |
| 199 | 7276 | TTR | −0.41 | −3.11 | 0.46 |
| 8 | 8503 | PIK3R3 | −0.41 | −2.92 | 0.58 |
| 198 | 1191 | CLU | −0.42 | −2.36 | 0.77 |
| 360 | 7405 | UVRAG | −0.43 | −3.07 | 0.55 |
| 335 | 115362 | GBP5 | −0.45 | −3.12 | 0.59 |
| 309 | 3320 | HSP90AA1 | −0.47 | −3.87 | 0.27 |
| 348 | 9500 | MAGED1 | −0.47 | −2.75 | 0.73 |
| 288 | 7419 | VDAC3 | −0.48 | −3.41 | 0.55 |
| 147 | 30011 | SH3KBP1 | −0.48 | −3.50 | 0.52 |
| 38 | 311 | ANXA11 | −0.49 | −3.31 | 0.61 |
| 358 | 3606 | IL18 | −0.50 | −3.29 | 0.64 |
| 327 | 6636 | SNRPF | −0.51 | −3.34 | 0.65 |
| 18 | 7299 | TYR | −0.52 | −2.99 | 0.73 |
| 353 | 3902 | LAG3 | −0.53 | −3.65 | 0.59 |
| 274 | 720 | C4A | −0.53 | −3.55 | 0.62 |
| 24 | 9133 | CCNB2 | −0.54 | −3.45 | 0.66 |
| 265 | 3446 | IFNA10 | −0.55 | −3.73 | 0.60 |
| 21 | 154 | ADRB2 | −0.56 | −4.77 | 0.24 |
| 68 | 59067 | IL21 | −0.58 | −4.29 | 0.51 |
| 266 | 3441 | IFNA4 | −0.58 | −3.18 | 0.74 |
| 284 | 174 | AFP | −0.60 | −3.61 | 0.69 |
| 230 | 64806 | IL25 | −0.64 | −3.74 | 0.70 |

The GeneID is found on NCBI website available at www.ncbi.nlm.nih.gov. More information about the gene can be found by accessing the NCBI website and entering the GeneID or Gene Symbol, for instance.

### REFERENCES

Bei, R., Masuelli, L., Palumbo, C., Modesti, M., and Modesti, A. (2009). A common repertoire of autoantibodies is shared by cancer and autoimmune disease patients: Inflammation in their induction and impact on tumor growth. Cancer Lett. 281, 8-23.

Brahmer, J. R., Tykodi, S. S., Chow, L. Q. M., Hwu, W.-J., Topalian, S. L., Hwu, P., Drake, C. G., Camacho, L. H., Kauh, J., Odunsi, K., et al. (2012). Safety and Activity of Anti-PD-L1 Antibody in Patients with Advanced Cancer. N. Engl. J. Med. 366, 2455-2465.

Buchbinder, E. I., and Desai, A. (2016). CTLA-4 and PD-1 Pathways: Similarities, Differences, and Implications of Their Inhibition. Am. J. Clin. Oncol. 39, 98-106.

Budde, P., Zucht, H.-D., Vordenba umen, S., Goehler, H., Fischer-Betz, R., Gamer, M., Marquart, K., Rengers, P., Richter, J., Lueking, A., et al. (2016). Multiparametric detection of autoantibodies in systemic lupus erythematosus. Lupus 25, 812-822.

Chiaruttini, G., Mele, S., Opzoomer, J., Crescioli, S., Ilieva, K. M., Lacy, K. E., and Karagiannis, S. N. (2017). B cells and the humoral response in melanoma: The overlooked players of the tumor microenvironment. Oncolmmunology 6, e1294296.

Ferlay, J., Soerjomataram, I., Dikshit, R., Eser, S., Mathers, C., Rebelo, M., Parkin, D. M., Forman, D., and Bray, F. (2015). Cancer incidence and mortality worldwide: sources, methods and major patterns in GLOBOCAN 2012. Int. J. Cancer 136, E359-386.

Gulley, J. L., Madan, R. A., Tsang, K. Y., Jochems, C., Marté, J. L., Farsaci, B., Tucker, J. A., Hodge, J. W., Liewehr, D. J., Steinberg, S. M., et al. (2014). Immune Impact Induced by PROSTVAC (PSA-TRICOM), a Therapeutic Vaccine for Prostate Cancer. Cancer Immunol. Res. 2, 133-141.

Halabi, S., Lin, C.-Y., Kelly, W. K., Fizazi, K. S., Moul, J. W., Kaplan, E. B., Morris, M. J., and Small, E. J. (2014). Updated Prognostic Model for Predicting Overall Survival in First-Line Chemotherapy for Patients With Metastatic Castration-Resistant Prostate Cancer. J. Clin. Oncol. 32, 671-677.

Kantoff, P. W., Gulley, J. L., and Pico-Navarro, C. (2017). Revised Overall Survival Analysis of a Phase II, Randomized, Double-Blind, Controlled Study of PROSTVAC in Men With Metastatic Castration-Resistant Prostate Cancer. J. Clin. Oncol. 35, 124-125.

Madan, R. A., Mohebtash, M., Arlen, P. M., Vergati, M., Rauckhorst, M., Steinberg, S. M., Tsang, K. Y., Poole, D. J., Parnes, H. L., Wright, J. J., et al. (2012). Ipilimumab and a poxviral vaccine targeting prostate-specific antigen in metastatic castration-resistant prostate cancer: a phase 1 dose-escalation trial. Lancet Oncol. 13, 501-508.

Manson, G., Norwood, J., Marabelle, A., Kohrt, H., and Houot, R. (2016). Biomarkers associated with checkpoint inhibitors. Ann. Oncol. 27, 1199-1206.

Palermo, G., Piraino, P., and Zucht, H.-D. (2009). Performance of PLS regression coefficients in selecting variables for each response of a multivariate PLS for omics-type data. Adv. Appl. Bioinforma. Chem. AABC 2, 57-70.

Robin, X., Turck, N., Hainard, A., Tiberti, N., Lisacek, F., Sanchez, J.-C., and Müller, M. (2011). pROC: an open-source package for R and S+ to analyze and compare ROC curves. BMC Bioinformatics 12, 77.

Spain, L., Diem, S., and Larkin, J. (2016). Management of toxicities of immune checkpoint inhibitors. Cancer Treat. Rev. 44, 51-60.

Topalian, S. L., Taube, J. M., Anders, R. A., and Pardoll, D. M. (2016). Mechanism-driven biomarkers to guide immune checkpoint blockade in cancer therapy. Nat. Rev. Cancer 16, 275-287.

Tusher, V. G., Tibshirani, R., and Chu, G. (2001). Significance analysis of microarrays applied to the ionizing radiation response. Proc. Natl. Acad. Sci. U.S.A. 98, 5116-5121.

1. A method of identifying a tumor-associated antigen (TAA) for prostate cancer comprising:
a) selecting a group of patients with prostate cancer and a group of patients who are healthy;
b) assaying the level of an autoantibody to an antigen in a sample from a patient in the group;
c) comparing the level of the autoantibody from the patient in the group or the group of patients with prostate cancer to the level of the autoantibody in the group of healthy patients; and
d) determining that the antigen is a TAA for prostate cancer if the level of the autoantibody to the antigen is statistically different between the group of patients with prostate cancer versus the group of healthy patients.

2. The method of claim 1, wherein the antigen is an antigen encoded by a gene listed in Table 1.

3. The method of claim 1, wherein the TAA is encoded by a gene listed in Table 2.

4. The method of claim 1, wherein the assaying comprises b1) contacting a portion of serum from the patient with a sample of an antigen immobilized onto a solid support.

5. The method of claim 4, wherein the solid support is a bead.

6. The method of claim 5, wherein the bead is a microsphere.

7. A method of identifying a tumor-associated antigen (TAA) as a marker for prostate cancer vaccination response comprising:
a) selecting a group of patients with prostate cancer who have been vaccinated with a vaccine effective to induce an immune response against a prostate cancer antigen and a group of patients with prostate cancer who have not been vaccinated with the vaccine;
b) assaying the level of an autoantibody to the antigen in a sample from each of the patients with prostate cancer who have been vaccinated;
c) comparing the level of the autoantibody to the antigen in each of the patients with prostate cancer who have been vaccinated to the level of the autoantibody in each of the patients with prostate cancer who have not been vaccinated; and
d) determining that the antigen is a TAA marker for prostate cancer vaccination response if the level of the autoantibody to the antigen is statistically different between the group of patients with prostate cancer who have been vaccinated versus the group of patients with prostate cancer who have not been vaccinated.

8. The method of claim 7, wherein the antigen is encoded by a gene listed in Table 3.

9. The method of claim 7, wherein the TAA marker for prostate cancer is encoded by a gene listed in Table 3.

10. The method of claim 7, wherein the assaying comprises b1) contacting a portion of serum from the patient with a sample of an antigen immobilized onto a solid support.

11. The method of claim 10, wherein the solid support is a bead.

12. The method of claim 11, wherein the bead is a microsphere.

13. A method of identifying and treating a prostate cancer patient with PROSTVAC therapy or for vaccination with a prostate antigen comprising:
a) determining the level of one or more antigens encoded by a gene listed in Table 4 having a positive value for r_in_PROSTVAC Progression-free survival;
b) assaying the level of one or more antigens in a sample from a prostate cancer patient;
c) comparing the level of the one or more antigens with an average level of the one or more antigens for a group of patients with prostate cancer who have not undergone PROSTVAC therapy; and
d) administering the PROSTVAC therapy, Ipilimumab, and/or the vaccination with a prostate antigen if the level of the one or more antigens in the patient is greater than the average level of the one or more antigens in the group of patients with prostate cancer.

14-18. (canceled)

19. A method of identifying and treating a prostate cancer patient with PROSTVAC therapy or for vaccination with a prostate antigen comprising:
a) determining the level of one or more antigens encoded by a gene listed in Table 4 having a negative value for r_in_PROSTVAC Progression-free survival;
b) assaying the level of one or more antigens in a sample from a prostate cancer patient;
c) comparing the level of the one or more antigens with an average level of the one or more antigens for a group of patients with prostate cancer; and
d) administering the PROSTVAC therapy, Ipilimumab, and/or the vaccination with a prostate antigen if the level of the one or more antigens in the patient is less than the average level of the one or more antigens in the group of patients with prostate cancer.

20-24. (canceled)

25. A method of monitoring the effectiveness of therapy in a prostate cancer patient previously treated with PROSTVAC vaccination or prostate antigen vaccination comprising:
a) determining the level of one or more antigens encoded by a gene listed in Table 4 having a negative value for r_in_PROSTVAC Progression-free survival by assaying a sample from a prostate cancer patient;
b) comparing the level of the one or more antigens with an average level of the one or more antigens for a group of patients with prostate cancer; and
c) determining that the PROSTVAC therapy is effective if the level of the one or more antigens in the patient is less than the average level of the one or more antigens in the group of patients with prostate cancer.

26. A method of monitoring the effectiveness of therapy in a prostate cancer patient previously treated with PROSTVAC vaccination or prostate antigen vaccination comprising:
a) determining the level of one or more antigens encoded by a gene listed in Table 4 having a positive value for r_in_PROSTVAC Progression-free survival by assaying the level of one or more antigens in a sample from a prostate cancer patient;
b) comparing the level of the one or more antigens with an average level of the one or more antigens for a group of patients with prostate cancer, and
c) determining that the therapy is effective if the level of the one or more antigens in the patient is greater than the average level of the one or more antigens in the group of patients with prostate cancer.

27-30. (canceled)

31. A method of identifying and treating a prostate cancer patient previously treated with PROSTVAC vaccination or prostate antigen vaccination comprising:
a) determining the level of one or more antigens encoded by a gene listed in Table 4 having a positive value for r_in_PROSTVAC Progression-free survival by assaying the level of one or more antigens in a sample from a prostate cancer patient;
b) comparing the level of the one or more antigens with an average level of the one or more antigens for a group of patients with prostate cancer; and
c) administering the therapy or the vaccination with a prostate antigen if the level of the one or more antigens in the patient is greater than the average level of the one or more antigens in the group of patients with prostate cancer,
wherein the therapy comprises one or more of Ipilimumab administration, prostate antigen vaccination, and PROSTVAC therapy.

32-36. (canceled)

37. A method of identifying and treating a prostate cancer patient previously treated with PROSTVAC vaccination or prostate antigen vaccination comprising:
a) determining the level of one or more antigens encoded by a gene listed in Table 4 having a negative value for r_in_PROSTVAC Progression-free survival by assaying the level of one or more antigens in a sample from a prostate cancer patient;
b) comparing the level of the one or more antigens with an average level of the one or more antigens for a group of patients with prostate cancer; and
c) administering the therapy if the level of the one or more antigens in the patient is less than the average level of the one or more antigens in the group of patients with prostate cancer, wherein the therapy comprises one or more of Ipilimumab administration, prostate antigen vaccination, and PROSTVAC therapy.

38-42. (canceled)

43. A method of monitoring the effectiveness of PROSTVAC therapy in a prostate cancer patient previously treated with PROSTVAC therapy or vaccination with a prostate antigen comprising:
a) determining the level of one or more antigens encoded by a gene listed in Table 4 having a negative value for r_in_PROSTVAC Progression-free survival by assaying the level of one or more antigens in a sample from a prostate cancer patient;
b) comparing the level of the one or more antigens with an average level of the one or more antigens for a group of patients with prostate cancer; and
c) determining that the PROSTVAC therapy is effective if the level of the one or more antigens in the patient is less than the average level of the one or more antigens in the group of patients with prostate cancer.

44. A method of monitoring the effectiveness of PROSTVAC therapy in a prostate cancer patient previously treated with PROSTVAC therapy or vaccination with a prostate antigen comprising:
a) determining the level of one or more antigens encoded by a gene listed in Table 4 having a positive value for r_in_PROSTVAC Progression-free survival by assaying the level of one or more antigens in a sample from a prostate cancer patient;
b) comparing the level of the one or more antigens with an average level of the one or more antigens for a group of patients with prostate cancer; and
c) determining that the PROSTVAC therapy is effective if the level of the one or more antigens in the patient is greater than the average level of the one or more antigens in the group of patients with prostate cancer.

45-48. (canceled)

49. A method of assessing overall survival of a patient who has been treated with PROSTVAC comprising:
a) determining the level of one or more antigens encoded by a gene listed in Table 5 having a positive value for r_in_PROSTVAC Overall Survival by assaying the level of one or more antigens in a sample from a prostate cancer patient; and
b) comparing the level of the one or more antigens with an average level of the one or more antigens for a group of patients with prostate cancer.

50. A method of monitoring the effectiveness of combined PROSTVAC with Ipilimumab therapy in a prostate cancer patient previously treated with combined PROSTVAC with Ipilimumab therapy comprising:
a) determining the level of one or more antigens encoded by a gene listed in Table 6 having a positive value for r-value Study.Day by assaying the level of one or more antigens in a sample from a prostate cancer patient;
b) comparing the level of the one or more antigens with an average level of the one or more antigens for a group of patients with prostate cancer; and
c) determining that the combined PROSTVAC with Ipilimumab therapy is effective if the level of the one or more antigens in the patient is greater than the average level of the one or more antigens in the group of patients with prostate cancer.

51. A method of monitoring the effectiveness of combined PROSTVAC with Ipilimumab therapy in a prostate cancer patient previously treated with combined PROSTVAC with Ipilimumab therapy comprising:
a) determining the level of one or more antigens encoded by a gene listed in Table 7 having a positive value for r_in_prostvac_ipi_Best.Response by assaying the level of one or more antigens in a sample from a prostate cancer patient;
b) comparing the level of the one or more antigens with an average level of the one or more antigens for a group of patients with prostate cancer; and
c) determining that the combined PROSTVAC with Ipilimumab therapy is effective if the level of the one or more antigens in the patient is greater than the average level of the one or more antigens in the group of patients with prostate cancer.

52. A method of assessing overall survival of a patient who has been treated with PROSTVAC and Ipilimumab comprising:
a) determining the level of one or more antigens encoded by a gene listed in Table 8 having a positive value for r_in_prostvac_ipi_Overall.Survival by assaying the level of one or more antigens in a sample from a prostate cancer patient; and
b) comparing the level of the one or more antigens with an average level of the one or more antigens for a group of patients with prostate cancer.

53. A method of monitoring for immune-related adverse events arising from combined PROSTVAC with Ipilimumab therapy in a prostate cancer patient previously treated with combined PROSTVAC with Ipilimumab therapy comprising:
a) determining the level of one or more antigens encoded by a gene listed in Table 9 having a positive value for Pearson'r by assaying the level of one or more antigens in a sample from a prostate cancer patient;
b) comparing the level of the one or more antigens with an average level of the one or more antigens for a group of patients with prostate cancer; and
c) determining that there is risk for an immune-related adverse event arising from combined PROSTVAC with Ipilimumab therapy if the level of the one or more antigens in the patient is greater than the average level of the one or more antigens in the group of patients with prostate cancer.
